(19) United States
(12) Patent Application Publication
(10) Pub. No.: US 2008/0215128 A1
(43) Pub. Date: Sep. 4, 2008

(54) ELECTRODE ARRANGEMENT FOR APPLYING ELECTRICAL SIGNALS TO THE SKIN OF AN ANIMAL

(76) Inventors: Christopher J. Rainey, Devon (GB); John P. Southgate, Devon (GB); Richard Nagle, London (GB); Andrea Moretti, London (GB)

Correspondence Address: DICKSTEIN SHAPIRO LLP, 1825 EYE STREET NW, Washington, DC, 20006-5403, US

(21) Appl. No.: 12/018,095

(22) Filed: Jan. 22, 2008

Related U.S. Application Data
(63) Continuation of application No. 11/802,201, filed on May 21, 2007, now abandoned.
Application No. 11/802,201 is a continuation-in-part of application No. PCT/GB2006/000315, filed on Jan. 31, 2006.
Application No. PCT/GB2006/000315 is a continuation-in-part of application No. 11/138,358, filed on May 27, 2005.

(30) Foreign Application Priority Data
Feb. 1, 2005 (GB) 0502070.6

(51) Int. Cl. A61N 1/04; A61F 13/00

(52) U.S. Cl. 607/152; 602/53

(57) ABSTRACT

An electrode arrangement for applying electrical signals to skin of an animal comprising a flexible electrically non-conductive substrate, at least one electrode provided on a first surface of the substrate for applying electrical signals to the skin when placed on the skin, at least one connector connected to a respective electrode for providing electrical signals to the respective electrode and an electrically non-conductive sealing arrangement connected to the substrate for sealing the substrate and the skin to prevent ingress of moisture to the at least one electrode and to the at least one connector at the connection to the respective electrode. The electrode, or each electrode, comprises a plurality of spaced electrically conductive elements to allow flexion of the electrode arrangement and electrically conductive material between the plurality of spaced electrically conductive elements to form a two-dimensional electrically conductive path across at least a portion of the first surface of the substrate.

This is a continuation of application Ser. No. 11/802,201, filed May 21, 2007, now abandoned, which is a continuation-in-part of International Application No. PCT/GB2006/000315, filed on Jan. 31, 2006, which claims priority from application Ser. No. 11/138,358, filed May 25, 2005 and GB 0502070.6, filed Feb. 1, 2005, the entire disclosures of which are incorporated by reference herein.

### FIELD OF THE INVENTION

The present invention relates to an electrode arrangement for applying electrical signals to the skin of an animal such as a human.

### BACKGROUND OF THE INVENTION

It is well documented that that the process of healing, growth and regeneration in living tissue is brought about by the flow of the body's own natural electrical current. Assisting the human body's natural healing tissue by applying electrical signals to the body in a form of therapy known as electrotherapy has been described in various publications. Electrotherapy is used in wound healing, pain treatment and muscle stimulation.

Effective electrotherapy requires the use of suitable electrodes or pads for administering electrical signals to the skin. For treatment to be successful, electrodes must be effectively electrically coupled to the skin. Accomplishing such an effective coupling is not evident when applying electrodes to uneven skin surface and curved body parts. Any such electrode device must therefore be sufficiently flexible to accommodate the curvaceous nature of the human body and to accommodate relative movement of patients skin during therapy, particularly the case when intended for multi-day wear. Precise placement of electrodes on the body is another important requirement influencing the outcome of treatment.

Various electrodes for application of electrical signals to the skin have been proposed.

U.S. Pat. No. 5,450,845 describes a medical electrode system which includes a flexible electrically conductive patch disposed on an electrically non-conductive backing material. The basic electro-conducting and electrical distribution portion of the electrode is washable and reusable. A separate disposable, adhesive, electrical conductive pad is used to couple this portion to the skin. One embodiment of the invention described uses a plurality of electrodes and a system for insuring proper placement of the electrode set on the body part.

U.S. Pat. No. 6,788,979 describes an electrical stimulation compress kit which allows for precise repeatable positioning of stimulation pads onto a body part. The device includes a flexible member for contacting a body part and hook/loop members for tightly supporting the flexible member against the body part.

### SUMMARY OF THE INVENTION

The present invention provides an electrode arrangement and a method for applying electrical signals to skin of an animal such as a human.

One aspect of the present invention provides an electrode arrangement for applying electrical signals to skin of an animal comprising a flexible substrate; at least one electrode provided on a first surface of the substrate for applying electrical signals to the skin when placed on the skin; electrically conductive gel applied to said at least one electrode for providing a conductive path from said at least one electrode to the skin; at least one connector lead for providing electrical signals to the respective electrode and having an electrically conductive part covered by a non-conductive covering; and an electrically non-conductive sealing arrangement connected to said substrate for preventing the ingress of moisture to said at least one electrode, said electrically conductive part of said at least one connector at the connection to said at least one electrode, and said electrically conductive gel.

In one embodiment, said electrically non-conductive sealing arrangement comprises a flexible sheet applied over a second surface of said substrate and comprising a first part covering said substrate and a second part extending laterally beyond said substrate, and an adhesive material applied to said second part of said flexible sheet for adhering said second part to said skin.

In one embodiment, said at least one connector lead is arranged to extend from said substrate across said adhesive material so that a seal is formed around said at least one connector lead when said second part is adhered to said skin.

In one embodiment, wherein said electrically non-conductive sealing arrangement comprises said substrate covering said at least one electrode, at least one portion of said substrate extending beyond said at least one electrode, and an adhesive material applied to said at least one portion of said substrate for adhering to said at least one portion of said substrate to said skin.

In one embodiment, said at least one connector lead is arranged to extend across said adhesive material so that a seal is formed around said at least one connector lead when said at least one portion of said substrate is adhered to said skin.

In one embodiment, wherein said at least one portion of said substrate extending beyond said at least one electrode has a width of 4 to 8 mm.

In one embodiment, said electrically non-conductive sealing arrangement is arranged to effectively seal under a pressure of up to 50-60 mmHg constant pressure for up to 7 to 9 days.

Another aspect of the present invention provides an electrode arrangement for applying electrical signals to skin of an animal comprising at least one flexible electrode for applying electrical signals to the skin when placed on the skin; electrically conductive gel applied to said at least one electrode for providing a conductive path from said at least one electrode to the skin; at least one connector lead connected to a respective said at least one electrode for providing electrical signals to the respective electrode and having an electrically conductive part covered by a non-conductive covering; and an electrically non-conductive sealing arrangement covering said at least one electrode for preventing the ingress of moisture to said at least one electrode, said electrically conductive part of said at least one connector at the connection to said at least one electrode, and said electrically conductive gel.

In one embodiment, said electrically non-conductive sealing arrangement is arranged to effectively seal under a pressure of up to 60 mmHg constant pressure for up to 7 to 9 days.

Another aspect of the present invention provides a wound treatment arrangement for treating a wound in the skin of a patient, the wound treating arrangement comprising a compression dressing arrangement applied to the limb of the patient and applying pressure thereto; and a flat electrode arrangement for applying electrical signals to said skin, said electrode arrangement comprising: a flexible electrically non-conductive substrate; and at least one electrode provided on a first region of a first surface of said substrate for applying electrical signals to the skin; wherein said at least one electrode is arranged on said skin adjacent to said wound under said compression dressing arrangement, said substrate includes an extended portion comprising at least one electrically conductive region, each electrically conductive region is in electrical contact with a respective electrode to form a respective connector to said electrode, and the at least one connector extends under and out from said compression dressing arrangement to enable electrical signals to be applied to said skin.

In one embodiment, an electrical generator apparatus is attached to said compression dressing arrangement and connected to said at least one connector for generating electrical signals to be applied to said skin.

In one embodiment, said at least one electrode comprises a plurality of interconnected electrically conductive elements to form a two-dimensional electrically conductive path across at least a portion of the first region of the first surface of the substrate.

In one embodiment, the plurality of interconnected electrically conductive elements are arranged in a matrix to allow flexion of the electrode arrangement

In one embodiment, the plurality of interconnected electrically conductive elements are arranged in a mesh pattern to allow flexion of the electrode arrangement

In one embodiment, electrically conductive gel is provided on said at least one electrode to provide an electrically conductive path to the skin.

In one embodiment, said at least one electrode comprises at least three electrodes and said at least one connectors comprises at least three respective connectors, wherein the electrical generator apparatus is adapted to switch current to flow between different electrodes of the at least three electrodes.

In one embodiment, said first region of said substrate has a part annular or crescent shape.

Another aspect of the present invention provides a pad for application to a wound to extend laterally across the wound, the pad comprising one or more elements of absorbent material for absorbing fluid exuded from the wound and distributed in a pattern laterally across the pad; and a plurality of portions of electrically insulating material interposed laterally between the elements of absorbent material to provide high resistance to current flow in at least one direction laterally across the pad.

In one embodiment, the portions of electrically insulating material are arranged in substantially parallel strips to provide high resistance laterally in at least one direction across the pad.

In one embodiment, the portions of electrically insulating material are arranged in a matrix to provide high resistance in any direction across the pad.

Another aspect of the present invention provides an electrode for applying current to the skin of a patient comprising a flat substrate having one of a substantially part annular shape and a substantially crescent shape; at least one electrode region applied to one surface of the substrate; at least one electrical connector connected to said at least one respective electrode; and an adhesive material for adhering the electrode to the skin of the patient.

Another aspect of the present invention provides an electrode for applying current to the skin of a patient comprising a flat substrate shaped as two substantially crescent shaped portions apically linked and having a concave inner circumferential edge and a convex outer circumferential edge, said outer circumferential edge having an indented region situated at the apically linked region between said substantially crescent shaped portions; at least one electrode region applied to one surface of the substrate; at least one electrical connector connected to said at least one respective electrode region; and an adhesive material for adhering the electrode to the skin of the patient.

In one embodiment, said substrate and said adhesive material forms a seal to seal the electrode against the ingress of moisture to a conductive path from said at least one electrical connector to the skin.

In one embodiment, said adhesive is arranged at a peripheral region of said substrate to provide a waterproof seal around said at least one electrode region, and said substrate is waterproof.

In one embodiment, a conductive gel is arranged on said at least one electrode region for conducting current between said at least one electrode region and the skin.

Another aspect of the present invention provides a method of treating a wound in the skin of a patient comprising placing a plurality of electrodes having one of a substantially part annular shape and a substantially crescent shape on the skin around the periphery of the wound so as to at least partly encircle the wound; and applying electrical current across the wound between said electrodes.

In one embodiment, said electrodes comprise a substrate shaped as two substantially crescent shaped portions apically linked and having a concave inner circumferential edge and a convex outer circumferential edge, each substantially crescent shaped portion having a substantially crescent shaped electrode portion applied thereto, said outer circumferential edge having an indented region situated at the apically linked region between adjacent crescent shaped portions, and the method includes placing a plurality of said electrodes close to the periphery of the wound by selecting to place either the inner circumferential edges of said plurality of said electrodes adjacent to the wound, and the indented region of the outer circumferential edges of said plurality of said electrodes adjacent to the wound.

Another aspect of the present invention provides an electrode arrangement for applying electrical signals to said skin, said electrode arrangement comprising a flexible electrically non-conductive substrate; at least one electrode provided on said substrate for applying electrical signals to the skin; electrically conductive gel provided on said at least one electrode to provide an electrical path to said skin; and retaining means for holding said gel in a plurality of sub regions across said at least one electrode so as to prevent said gel from moving from a sub region laterally under pressure.

In one embodiment, said retaining means comprises a plurality of walls of flexible non-conductive material extending from said at least one electrode in a two dimensional pattern over said at least one electrode, said walls define said sub regions, and said gel is provided on said at least one electrode between said pattern of walls.

Another aspect of the present invention provides an electrode arrangement for applying electrical signals to said skin, said electrode arrangement comprising a flexible electrically non-conductive substrate; at least one electrode provided on said substrate for applying electrical signals to the skin; a plurality of walls of flexible non-conductive material extending from said at least one electrode in a two dimensional pattern over said at least one electrode; and electrically conductive gel provided on said at least one electrode between said pattern of walls to provide an electrical path to said skin, said gel being laterally contained by said pattern of walls.

In one embodiment, said gel is highly resistive to overcome skin resistance.

Another aspect of the present invention provides a wound treatment arrangement for treating a wound in the skin of a patient, the wound treating arrangement comprising a compression dressing arrangement applied to the limb of the patient and applying pressure thereto; and a flat electrode arrangement for applying electrical signals to said skin, said electrode arrangement comprising: a flexible electrically non-conductive substrate; and at least one electrode provided on said substrate for applying electrical signals to the skin; wherein said electrode arrangement is arranged on said skin adjacent said wound under said compression dressing arrangement, and a high resistance is connected in series with said at least one electrode.

In one embodiment, the high resistance comprises a highly resistive gel provided on a surface of said at least one electrode.

In one embodiment, the high resistance comprises a plurality of elements of high resistivity distributed across at least a part of the surface of said at least one electrode for conduction of the electrical signal from said at least one electrode to the skin, wherein the elements of high resistivity are interspaced by portions of relatively electrically insulating material such that the elements of high resistivity are electrically insulated from one another.

In one embodiment, the elements of high resistivity comprise highly resistive gel.

In one embodiment, the portions of relatively electrically insulating material comprise rubber.

Another aspect of the present invention provides an electrode arrangement for applying electrical signals to said skin, said electrode arrangement comprising a flexible electrically non-conductive substrate; at least one electrode provided on said substrate for applying electrical signals to the skin; and a layer of electrically conductive gel lying over said at least one electrode and extending beyond said at least one electrode to cover a boundary region of said substrate for adhering said electrode arrangement to the skin and for providing an electrical path from said at least one electrode to the skin, wherein the thickness of said layer of gel is substantially smaller than the lateral length of said boundary region extending beyond said at least one electrode.

In one embodiment, said substrate and said gel form a seal around said electrode to prevent the ingress of water to said at least one electrode.

In one embodiment, at least one electrical connector lead is connected to said at least one electrode, wherein said substrate and said gel is adapted to seal said skin under said gel, said electrode, and said at least one connector lead in the region of connection to said at least one electrode to prevent the ingress of water to an electrical path from said connector to said skin.

In one embodiment, said boundary portion has a length of 4-8 mm. In one embodiment, the formed seal is arranged to effectively seal under a pressure of 50-60 mmHg constant pressure for up to 7 to 9 days.

Any aspect of the invention can be used in conjunction with another aspect of the invention.

### BRIEF DESCRIPTION OF THE DRAWINGS

Embodiments of the invention will now be described with reference to the accompanying drawings, in which:

FIG. 1 is a partial cross sectional view of an electrode arrangement according to a first embodiment of the present invention;

FIG. 2 is a schematic diagram of the electrode arrangement according to the first embodiment of the present invention;

FIG. 3 is a partial cross sectional view of an electrode arrangement according to a second embodiment of the present invention;

FIG. 4 is a schematic diagram of the electrode arrangement according to the second embodiment of the present invention;

FIG. 5 is a schematic diagram of an electrode arrangement according to a third embodiment of the present invention;

FIG. 6 is a schematic diagram of an area of treatment showing electrode arrangements of FIG. 5 placed around a wound;

FIG. 7 is a schematic diagram of an area of treatment showing electrode arrangements of FIGS. 1 and 2 disposed around a wound;

FIGS. 8A and 8B are partial cross sectional diagrams of electrode arrangements according to a fourth embodiment of the invention;

FIG. 9 is a schematic diagram of an area of treatment showing four electrode arrangements of any embodiment disposed around a wound;

FIG. 10 is a schematic diagram of an area of treatment according to an embodiment of the invention;

FIG. 11A is a schematic diagram of a device for generating electrical current according to an embodiment of the invention;

FIG. 11B is a schematic diagram of a device for generating electrical current according to an alternative embodiment of the invention;

FIG. 12 is a schematic diagram of an electrode arrangement according to a fifth embodiment of the invention;

FIG. 13 is a schematic diagram of an electrode arrangement according to a sixth embodiment of the invention;

FIG. 14 is a schematic view of an electrode arrangement according to a seventh embodiment of the invention, placed on an area of treatment;

FIG. 15 is a schematic view of an electrode arrangement according to an eighth embodiment of the invention;

FIG. 16 is a schematic diagram of an area of treatment showing the electrode arrangements of FIG. 15 placed around a wound;

FIG. 17 is a schematic diagram of an electrode arrangement according to a ninth embodiment of the invention;

FIGS. 18A and 18B are top and bottom views respectively of an electrode design according to a tenth embodiment of the present invention;

FIGS. 19A and 19B are top and bottom views respectively of an electrode design according to an eleventh embodiment of the present invention;

FIGS. 20A to E illustrate various placements of the electrodes of the tenth or eleventh embodiments of the invention around a wound;

FIG. 21 is a cross sectional diagram of an electrode arrangement according to a twelfth embodiment of the invention;

FIG. 22 is a schematic diagram of the electrode arrangement of FIG. 21 connected to another electrode arrangement;

FIG. 23 is a schematic diagram of an electrode arrangement according to a thirteenth embodiment of the invention;

FIG. 24 is a partial cross sectional diagram of an electrode arrangement according to a fourteenth embodiment of the invention:

FIG. 25 is a partial cross-sectional diagram of a pad for use with one or more electrode arrangements according to any of the embodiments of the invention;

FIG. 26 is a schematic diagram of a treatment area showing the electrode arrangement of FIG. 23 disposed around a wound; and

FIG. 27 is a schematic diagram of an electrode arrangement in which the electrically conductive gel provides adhesion of the electrode, a conductive path and sealing.

### DESCRIPTION OF THE EMBODIMENTS

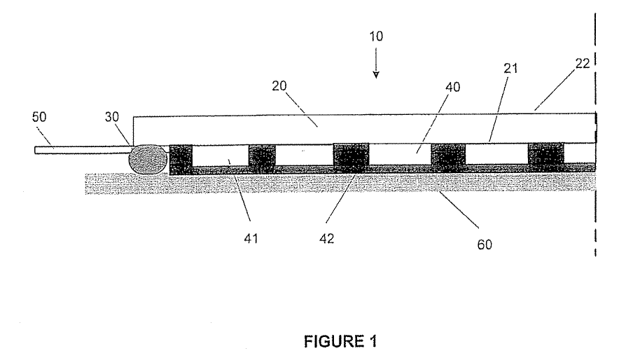

FIG. 1 is a partial cross sectional view of an electrode arrangement 10 placed on the skin 60 of a human patient according to a first embodiment of the present invention. The electrode arrangement comprises a flexible electrically non-conducting printed circuit board 20 having a first surface 21 and a second surface 22 opposed to the first surface, electrically non-conductive sealing gel 30, an electrode 40 for applying electrical signals to the skin 60 when placed in contact with the skin and an electrically conductive lead 50 for supplying electrical signals to the electrode 40. The conductive lead 50 is formed of a conductive core and an insulating covering to avoid stray currents and short circuits. The electrode 40 is formed from partial etching of a layer of electrically conductive material, such as gold plated copper, on the first surface 21 of the printed circuit board 20 and comprises electrically conductive tracks 41 with gaps there between. Electrically conductive gel 42 is placed in the gaps between the electrically conductive tracks 41 and over the surface of the electrically conductive tracks 41. The electrically non-conductive gel 30 is waterproof and is placed around the edges of the first surface 21 of the printed circuit board 20 and around the electrically conductive lead 50 adhering the printed circuit board 20 to the skin 60 preventing ingress of moisture such as sweat, urine or blood to the electrode 40 and ingress of moisture to the connection between the electrically conductive lead 50 and the electrode 40 and the electrically conductive gel 42 which may lead to short circuits, stray currents and other undesirable effects. The electrode arrangement is sealed by the substrate 20, the insulating covering on the conductive lead 50, and the sealing gel 30 to prevent ingress of any fluid to the electrical path from the conductive lead 50 to the skin 60. The components of the electrode arrangement are washable so that they may be reused if required.

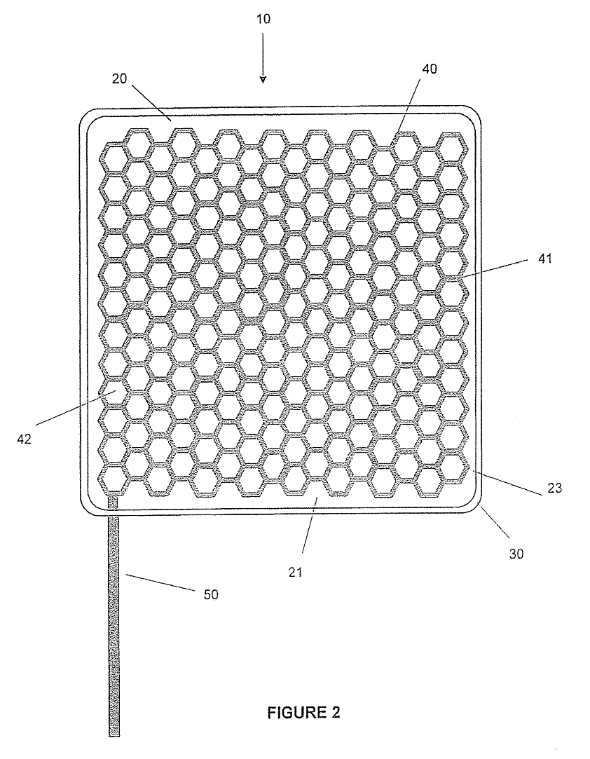

FIG. 2 is a schematic plan view of the electrode arrangement 10 showing the first surface 21 of the printed circuit board 20. The electrically conductive tracks 41 are arranged in the form of a honeycomb mesh structure with an electrical connector 50 which can be formed from an extended portion of the printed circuit board 20. The electrically conductive gel 42 is spread over the first surface 21 of the printed circuit board 20 into the gaps between the electrically conductive tracks 41 thereby forming further electrically conductive connections between the electrically conductive tracks 41, enhancing electrical conductivity across the surface of the electrode and providing a electrically conductive path between the electrode 40 and the skin. The sealing gel 30 is placed on a non-conductive portion 23 at the edges of the first surface 21 of the printed circuit board 20 in the form of a strip around the periphery of the printed circuit board 20. The printed circuit board 20 is fabricated from any suitable substrate such as a flexible polyester film. The combination of the honeycomb mesh pattern of electrically conductive tracks 41 and electrically conductive gel 42, with the polyester film printed circuit board provides an electrode arrangement which is flexible in a direction perpendicular to the plane of the electrode arrangement. This flexibility allows the electrodes 40 to make good electrical contact with the uneven or curvaceous surface of the skin when placed on the skin 60 with the first surface 21 of the printed circuit board 20 placed facing the skin 60. Furthermore this arrangement helps to eliminate hot spots resulting from uneven electrical-skin contact.

It should be appreciated that the gaps between the electrically conductive tracks 41 need only be sufficiently large to permit flexion of the printed circuit board.

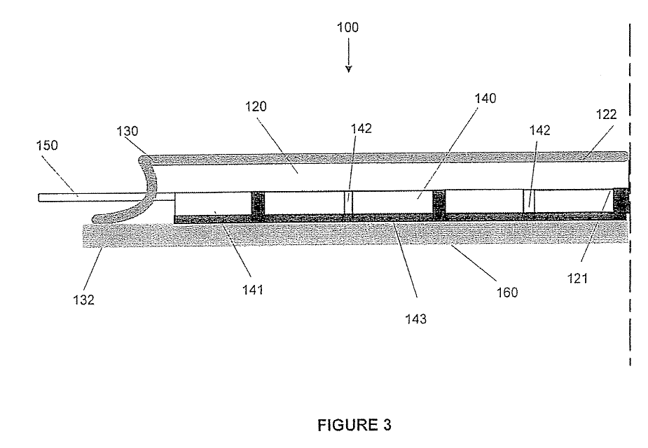

FIG. 3 is a partial cross sectional view of an electrode arrangement 100 placed on the skin 160 of a patient according to a second embodiment of the invention comprising a flexible electrically non-conducting printed circuit board 120 having a first surface 121 and a second surface 122 opposed to the first surface 121, a waterproof sealing pad 130 to prevent ingress of moisture to any exposed conductive elements of the electrode arrangement, an electrode 140 for applying electrical signals to the skin when placed on the skin, and a connector 150 for supplying electrical signals to the electrode 140. The electrode 140 comprises electrically conductive elements 141 and flexible electrically conductive links 142. Electrically conductive gel 143 is applied to the electrode 140 to provide an electrically conductive path between the electrode 140 and the skin. The sealing pad 130 is placed over the second surface 122 of the printed circuit board 120. The outer edges 132 on one surface of the sealing pad 130 have adhesive properties and the sealing pad is positioned such that the edges 132 of the sealing pad extend beyond the edges of the printed circuit board 120 in all directions and adhere the printed circuit board to the skin. The sealing pad 130 may be any type of waterproof adhesive material of the kind generally used in medicine.

The sealing pad can be a waterproof, oxygen permeable material, such as, for example Opsite™ film dressing from Smith and Nephew, Tegaderm™ dressing from 3M™, Bioclusiv™ dressing from Johnson and Johnson, Dermaview™ dressing from Dermarite or any other suitable wound dressing or adhesive material which is waterproof but oxygen permeable. In this way moisture is prevented from penetrating the dressing while oxygen which plays a part in wound healing can reach the wound. It will be understood that in this case a printed circuit board which is oxygen permeable can be used.

Although in this embodiment the sealing pad 130 and the printed circuit board 120 are shown as separate layers, it will be apparent that these can be formed of the same material and hence form a single material layer. In this arrangement, a region of the substrate of the printed circuit board extends beyond the region of the electrode 140 in a similar manner to the first embodiment and the substrate is flexible at the peripheral regions so as to flex toward the skin to reduce the thickness of gel or adhesive material needed to seal the substrate to the skin.

In any of the embodiments of the invention, the dimensions and adhesive properties of the peripheral region at which the substrate or sealing pad is adhered to the skin sufficient effectively seal under a pressure of up to 60 mmHg constant pressure for up to 7 to 9 days. Thus, the electrode arrangement can be worn for a significant treatment period under a compression bandage to enable simultaneous treatment of wounds such as diabetic food ulcers with electrical stimulation and compression dressings including bandages, tubing, socks etc. In one embodiment the dimensions of the peripheral region at which the substrate or sealing pad is adhered to the skin is between 4 and 8 mm.

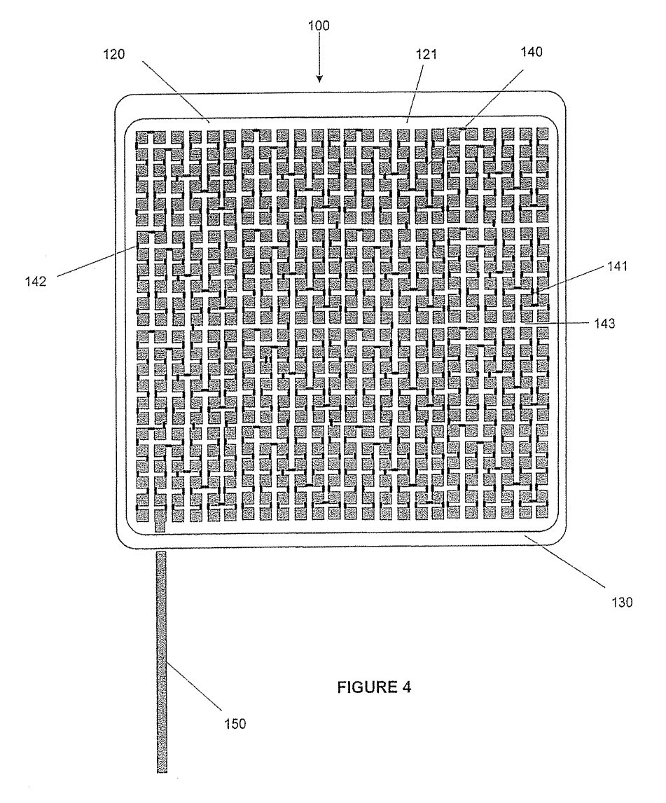

FIG. 4 is a schematic plan view of the electrode arrangement 100 according to the second embodiment of the invention showing the first surface 121 of the printed circuit board 120. The electrically conductive elements 141 are square shaped and are etched out of a layer of conductive material such as gold plated copper on the first surface 121 of the printed circuit board 120 in a matrix and are connected to an electrical connector lead 150 which can be formed of an extended portion of the printed circuit board 120. Flexible electrically conductive links 142 are disposed between some of the electrically conductive elements 141 to form a bi-dimensional electrically conductive path across a portion of the first surface 121 of the printed circuit board 120. The printed circuit board 120 is fabricated from a polyester film. The combination of the matrix of electrically conductive elements 141 and the flexible electrically conductive links 142, with the polyester film printed circuit board 120 provides an electrode arrangement which is flexible in a direction perpendicular to the plane of the electrode arrangement. Electrically conductive gel 143 is applied to the electrode 140 to provide an electrically conductive path between the electrode and the skin. Such an arrangement provides an electrode arrangement which makes good contact with uneven surfaces such as the skin.

Although in this embodiment a polyester film is used as the printed circuit board, in alternative embodiments any flexible PCB material may be used.

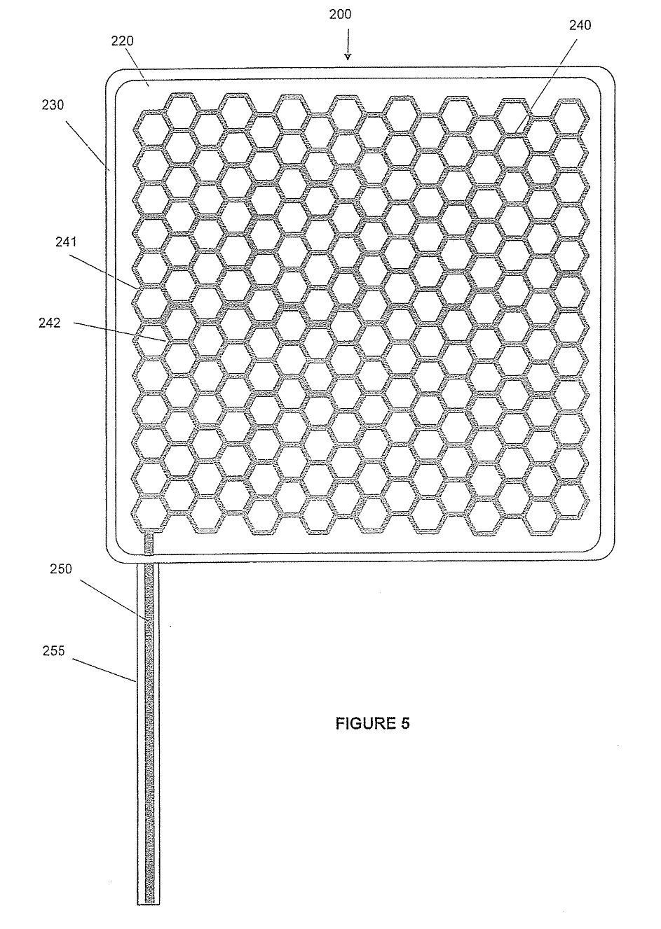

FIG. 5 is a schematic plan view of an electrode arrangement 200 according to a third embodiment of the present invention. The electrode arrangement 200 comprises a flexible electrically non-conducting printed circuit board 220 with an extended portion 255 of length 5 cm to 50 cm, an electrode 240 comprising a honeycomb mesh pattern of electrically conductive tracks 241 and electrically conductive gel 242. Waterproof sealing gel 230 is placed around the edges of the printed circuit board to provide a seal between the electrode arrangement and skin preventing the ingress of moisture. The extended portion 255 of the printed circuit board 220 carries a flexible electrical connector 250 which when connected to an electrical current generator can carry current to the electrode 240. The portion of the flexible electrical connector on the extended portion is electrically insulated by an insulating layer on top of the flexible electrical connector 250. The extended portion provides a flat lead to the electrode which will not indent the skin of a patient or cause discomfort and will not appear as a raised region or bulge when covered with a bandage or other type of medical dressing. It will be understood that the length of the extended portion is such that it extends from the wound area along the leg of the patient to provide a connection between the electrode arrangement located in the area of the wound under a dressing and an electrical generator located outside the dressing.

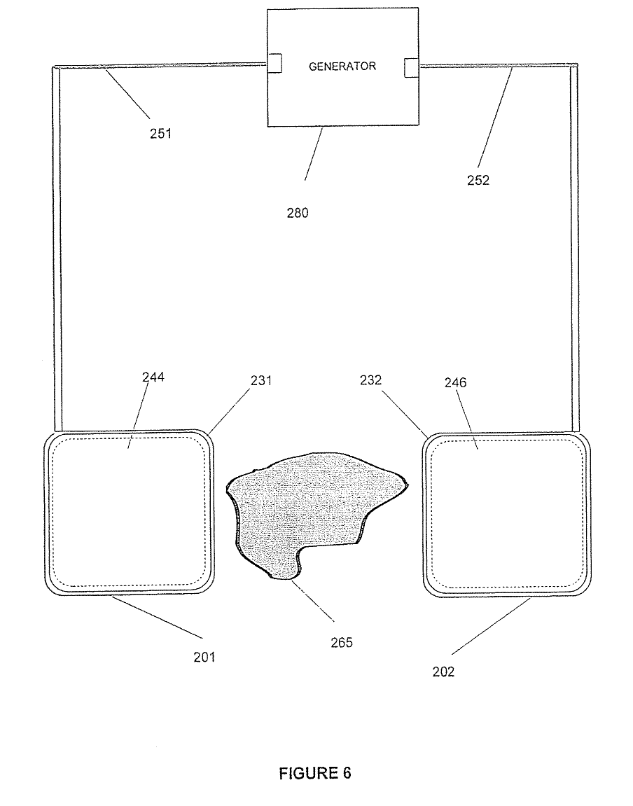

FIG. 6 is a schematic plan view of an area of treatment in which electrode arrangements according to the third embodiment of the invention are placed around a wound 265 of a human patient. Electrode arrangements 201 and 202 are placed on the skin on opposite sides of the wound 265 with the first surface of the printed circuit board of each electrode arrangement arranged to face the skin. The edges of the printed circuit boards of 201 and 202 on which adhesive sealing gel 231 and 232, respectively, is applied, are pressed against the skin in order to adhere the electrode arrangement to the surface of the skin. This arrangement creates a seal preventing the ingress of moisture to any exposed conductive components of the electrode arrangements. The electrode arrangements 201 and 202 are connected to an electrical generator circuit 280 via connectors 251 and 252, respectively, which are provided on the extended portions of printed circuit boards 201 and 202 respectively as for the previous embodiment. Electrical signals generated by the electrical generator 280 pass through the connectors 251 and 252 to electrodes 244 and 246, respectively (shown as dotted areas). From the electrodes 244 and 246 pressed against the skin of the patient, the electrical signals are administered to the skin of the patient around the wound. Such an arrangement promotes wound healing by passing electrical current between the electrodes through regenerative tissues under the wound.

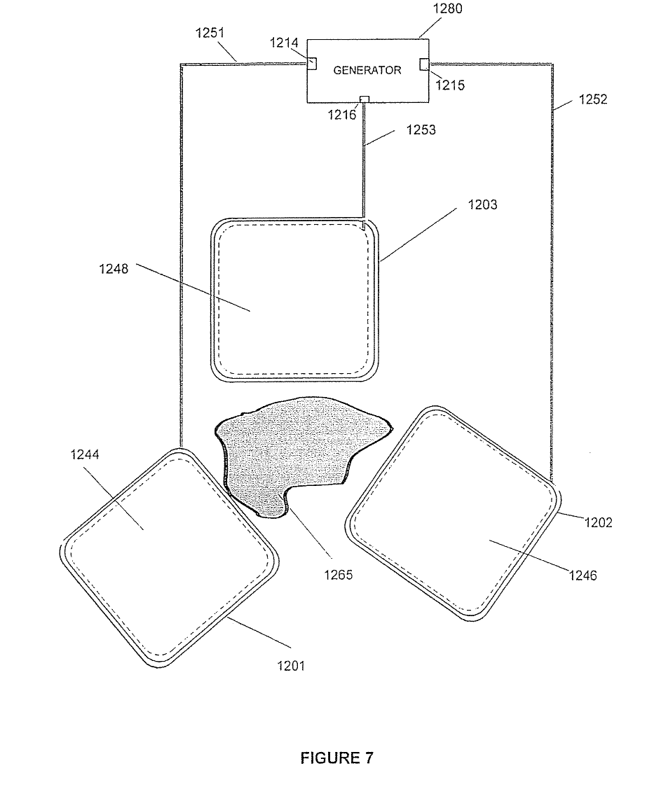

FIG. 7 is a schematic view of an area of treatment in which electrode arrangements according to the first embodiment of the invention are placed around a wound. Electrode arrangements 1201, 1202 and 1203 are arranged around a wound 1265. The first surface of the printed circuit board of the electrode arrangements are arranged to face the skin of the patient. The outer edges of the printed circuit boards of electrode arrangements 1201, 1202 and 1203 on which adhesive gel is applied is pressed against the skin to seal the electrode arrangement to the skin and prevent the ingress of moisture to any exposed conductive components. Connecting leads 1251, 1252 and 1253 are connected between an electrical generator circuit 1280 and to electrode arrangements 1201, 1202 and 1203, respectively. Connector 1251 supplies electrical signals from the electrode output port 1214 of generator 1280 to electrode 1244, electrical connector 1252 supplies signals from the electrode output port 1215 of generator 1280 to electrode 1246 and electrical connector 1253 supplies signals from the electrode output port 1216 of generator 1280 to electrode 1248. Electrical current can therefore be applied between different electrodes, thereby passing through different paths under the wound. For example, electrical current may be applied to pass from electrode 1244 to electrode 1248 or from electrode 1244 to electrode 1246. Furthermore, the direction of the current flow may be reversed so that current flows from electrode 1248 to electrode 1244, or from electrode 1246 to electrode 1244 Alternatively, electrical current may be applied to pass from electrode 1248 to electrodes 1244 and 1246 simultaneously, or alternatively, to pass from electrodes 1244 and 1246 to electrode 1248. It may be appreciated that it is possible to apply the current to flow between many combinations of different electrodes.

Although, in this embodiment three electrodes are used, any number of electrodes may be placed around the wound to provide finer angular steps between electrode positions and resulting in more complex current profile patterns across the area of treatment if required. Current may be applied to flow between different electrodes simultaneously or in a sequence depending on the treatment required. Furthermore, the electrical generator 1280 may be programmed to provide a pre-determined sequence of current profiles through different paths across the treatment area.

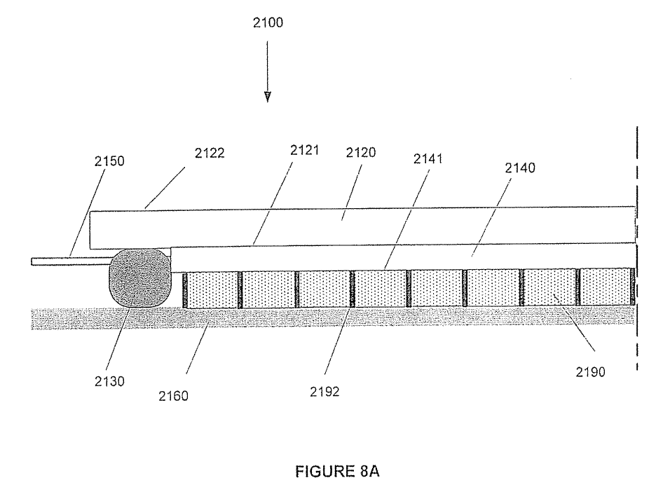

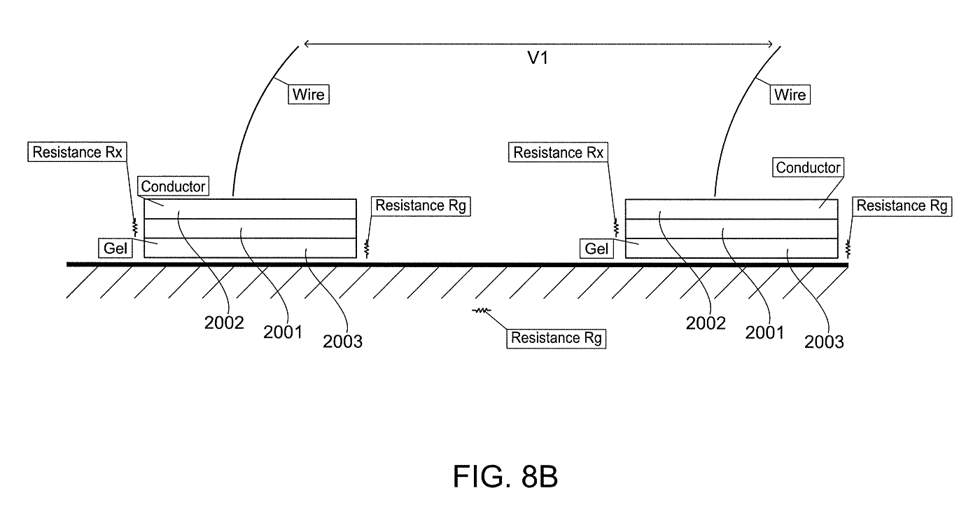

FIG. 8A is a partial cross-sectional diagram of an electrode arrangement 2100 according to a fourth embodiment on the invention disposed on the surface of the skin 2160 of a patient. The electrode arrangement comprises a flexible electrically non-conductive substrate 2120 having a first surface 2121 facing towards the skin when the electrode arrangement is placed on the skin and a second surface 2122 opposed to the first surface, electrically non-conductive sealing gel 2130, an electrode 2140 for applying electrical signals to the skin 2160, and an electrically conductive lead 2150 for supplying electrical signals to the electrode 2140. The electrode 2140 has a first surface 2141 facing towards the skin when the electrode arrangement is placed on the skin. A plurality of interconnected flexible elements 2192 are disposed on the first surface 2141 of the electrode 2140. The flexible elements are arranged in a honeycomb mesh structure across the first surface 2141 of the electrode 2140 and each flexible element extends from the first surface 2141 of the electrode 2140 towards the surface of the skin 2160 when the electrode arrangement is placed on the skin. Portions of electrically conductive gel 2190 are disposed on the first surface 2141 of the electrode 2140 between the flexible elements 2192. The flexible elements prevent the migration of the gel laterally and hence the reduction if the thickness of the gel at locations across the electrode. The electrode arrangement is flexible and will follow the contours of the skin without the thickness of the gel varying to accommodate the contours of the skin. Consequently, differences in resistances of the electrode and the skin across the surface of the electrode will be small. The result will be a more even distribution of current across the surface of the electrode.

The flexible elements can be formed of electrically conductive material to assist in the conduction of the current to the skin. When the electrode arrangement is used on a contoured part of the skin there is a possibility of poor contact with the skin in regions of the electrode. This can cause ‘hot spots’ of high current. The flexible elements can thus be made of insulating material to prevent lateral currents between regions of gel and hence reduce hot spots in such circumstances.

The gel can have a high resistance to provide a high resistive path distributed over the surface of the electrode which renders any resistance of the skin and the electrode negligible in comparison to the resistance of the gel.

Although in this embodiment, portions of electrically insulating rubber are used it may be understood that any electrically insulating material providing flexibility may be used. Furthermore, although gel of high electrical resistivity is used in this embodiment, in alternative embodiments any material of high electrical resistivity may be used. In further embodiments of the invention a continuous layer of highly resistive gel without interspacing by portions of electrically insulating material may be used.

Although in this embodiment the portions of electrically insulating rubber are arranged in the form of a honeycomb mesh structure, in alternative embodiments of the invention, the portions of electrically insulating rubber may be arranged in any mesh or grid structure preventing a path of electrical conductivity from being created through the mesh structure and preventing lateral movement of the gel. Any structure of walls or containers can be used to contain the gel in discrete locations.

While in this embodiment, the electrode arrangement includes a sealing arrangement it will be understood that in alternative embodiments the sealing arrangement need not be present.

In alternative embodiments of the invention the effect of a more even distribution of current may be achieved by connecting a high resistance in series with the electrode. FIG. 8B illustrates such an alternative arrangement in which a high resistance layer 2001 is provided in parallel with and between the conductive electrode layer 2002 and the gel layer 2003. The conductive electrode layer 2002 can be a solid layer or a grid or matrix of conductive material applied to the high resistance layer 2001.

In a conventional electrode arrangement, or in any one of the first to third embodiments for example, the total current being delivered will be determined by the forcing voltage “V₁” between the connection to the electrodes, the impedance of the gel “Rg”, the impedance of patients body between the electrodes “Rb” such that the current I=V₁/(Rg+Rb+Rg).

However the impedance of the gel “Rg” is an average impedance made up of many resistors in parallel rg₁, rg₂ . . . rgₙ thus (1/Rg=1/rg₁+1/rg₂+ . . . 1/rgₙ)

If the resistors are equal then rg₁=rg₂ . . . =rgₙ

If there were 100 elements distributed over the surface then each element=Rg*100

Due to variation in thickness of the gel, manufacture, distortion of the gel due to pressure or stretch or variations in skin resistance, each of the elements making up the average impedance will also be unequal. This will give rise to variations in impedance across the pad.

As illustrated in FIG. 8B, if a further resistor is introduced in the path of each element rx that is large in comparison with Rg such that (1/Rx=1/rx₁+1/rx₂+ . . . 1/rxₙ) and hence the elements rg₁=rg₂ . . . =rgₙ then the variation in current across the electrode will be determined by the impedance then I=Vᵢ/(Rg+Rx+Rb+Rg+Rx). If Rx>>Rb and Rx>>Rg and the equation simplifies to I˜V1/2Rx. Therefore, providing that lateral currents are minimal, the effect of skin and gel properties can be marginalised.

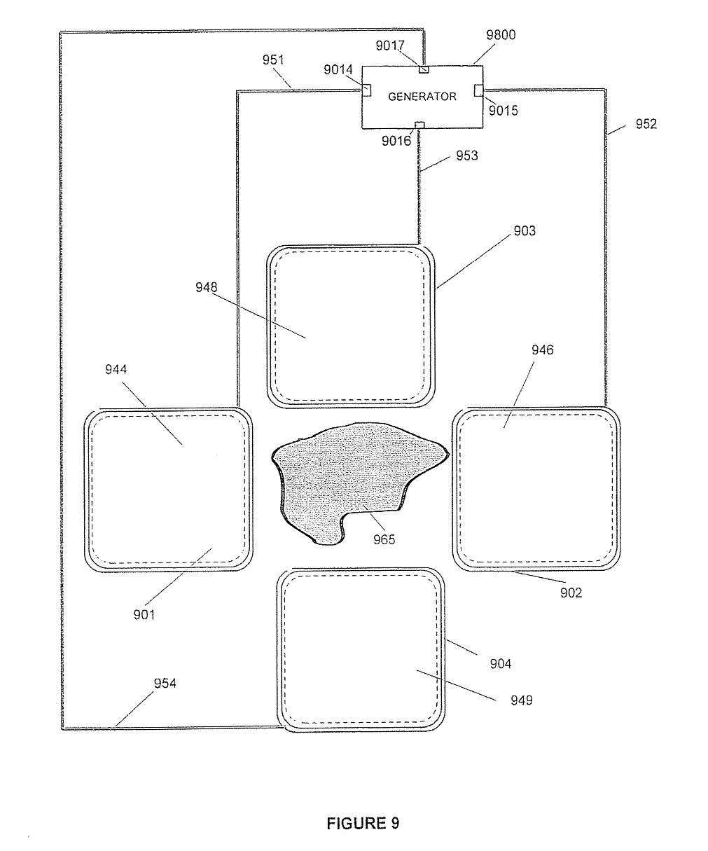

FIG. 9 is a schematic view of an area of treatment in which four electrode arrangements according to the fourth embodiment of the invention are placed around a wound. Electrode arrangements 901, 902, 903 and 904 are arranged around a wound 965. The first surface of the printed circuit board of the electrode arrangements are arranged to face the skin of the patient. The outer edges of the printed circuit boards of electrode arrangements 901, 902, 903 and 904, on which adhesive gel is applied, is pressed against the skin to seal the electrode arrangement to the skin and to prevent the ingress of moisture to any exposed component parts. Connecting leads 951, 952, 953 and 954 are connected between an electrical generator circuit 9800 capable of generating a bi-directional constant current, and electrode arrangements 901, 902, 903 and 904, respectively. Connector 951 supplies electrical signals from the electrode output port 9014 of generator 9800 to electrode 944, electrical connector 952 supplies signals from the electrode output port 9015 of generator 9800 to electrode 946, electrical connector 953 supplies signals from the electrode output port 9016 of generator 9800 to electrode 948, and electrical connector 954 supplies electrical signals from the electrode output port 9017 of generator 9800 to electrode 949. Electrical current can therefore be applied to pass under the wound between different electrodes. A maximum current limit can be set on the electrical current generated by the electrical generator 9800 for each pair of electrodes. Any combination of electrodes may be used thereby forming a matrix of current paths under the wound and ensuring a more even distribution of current. For example electrical current may be applied to pass from electrode 948 to electrode 949 and from electrode 946 to electrode 944. The direction of the current passing between one or both of these pairs of electrodes may be reversed. Alternatively, electrical current may be applied to pass from electrode 948 to electrode 946 and from electrode 944 to electrode 949. Again, the direction of the current passing between one or both of these pairs of electrodes may be reversed. It may be appreciated that it is possible to apply current to flow between many combinations of different electrodes. The current may be applied to flow between different electrodes simultaneously or sequentially.

In alternative embodiments of the invention the current may be applied by feeding each electrode current though a resistor having a resistance substantially greater than the resistance of the skin and the electrode. Such an arrangement renders any differences in resistances of the electrode and the skin at different points across the surface of the electrode insignificant and thus has the effect of providing a more even distribution of current.

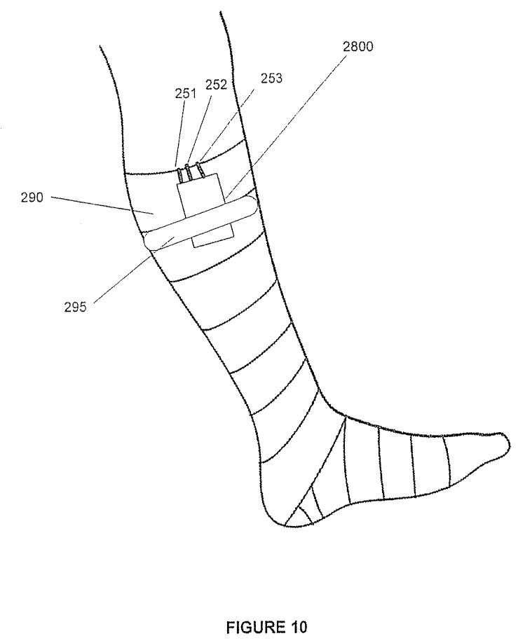

FIG. 10 schematically illustrates the area of treatment of FIG. 7 covered by a compression bandage 290. Electrical generator 2800 is located outside the bandage 290 and attached to the leg by adhesive medical tape 295. Electrical interface between the electrical generator 2800 and electrodes is made through connectors 251, 252 and 253. Any previously described electrode arrangement embodiment can be used in this arrangement. The electrode arrangements formed from flat printed circuit boards in which the connectors are formed of extensions of a printed circuit board are particularly suited for use under compression dressings because of their low profile which reduces discomfort to the wearer and the likelihood of causing further wounds in susceptible patients.

Any of the electrode arrangements of the present invention can be used under a compression dressing as illustrated in FIG. 10. If sealing of the electrode arrangement is required, the arrangement should be capable of withstanding the pressure conditions experienced under the dressing for the period of application of the dressing on the patient. This is typically up to 50-60 mmHg of pressure constantly applied over a period of up to 7 to 9 days. Where a sealing gel or adhesive region is provided around the periphery of the electrode arrangement, the length of the boundary region providing the sealing and adhesion should be 4-8 mm.

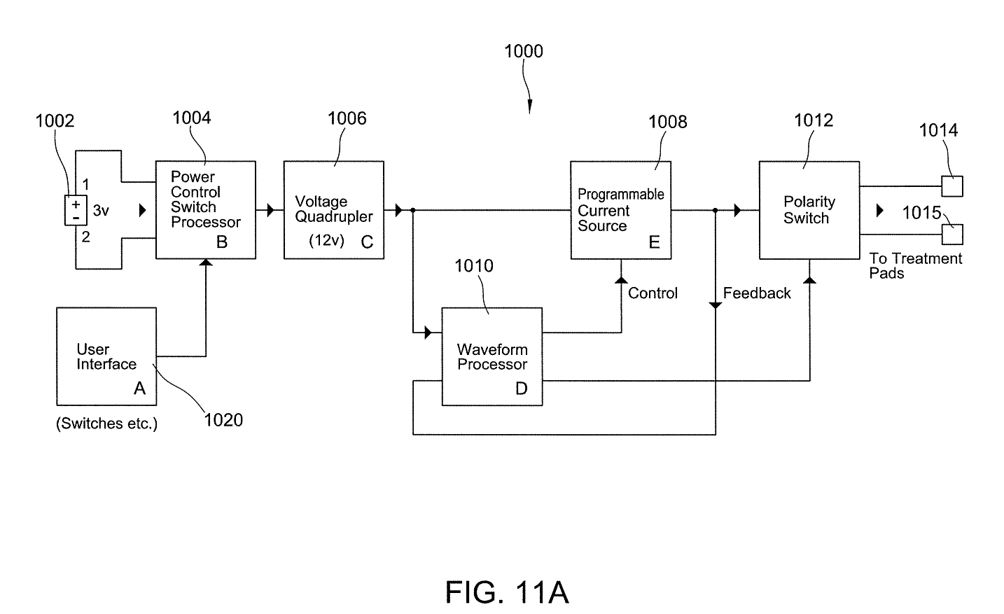

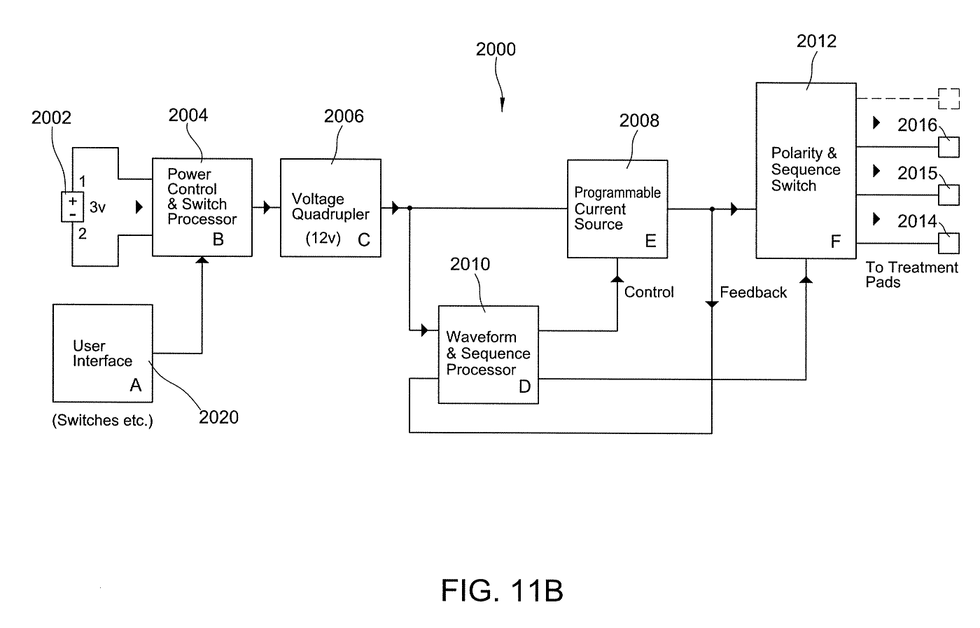

A schematic diagram of an electrical generator circuit according to an embodiment of the invention is shown in FIG. 11A. The electrical generator circuit 1000 comprises a battery 1002, a power control processor 1004, a voltage multiplier 1006, a programmable current source 1008, a waveform processor 1010, a polarity switch 1012, a user interface 1020, electrode ports 1014 and 1015. The device 1000 can be connected to electrode arrangements through electrode ports 1014 and 1015.

Power control processor 1004 can activate the generation of current waveforms through user interface 1020 or automatically at predetermined times. Battery 1002 is a lithium coin cell type and runs the power control processor 1004 in low power mode. When current is required, power is supplied to the voltage multiplier 1006.

In this embodiment voltage multiplier 1006 is a dc-dc converter and multiplies the input voltage using a charge pump circuit. The charge pump circuit is intrinsically power and voltage limited thereby providing a “fail-safe” operation. The voltage multiplier 1006 supplies a pumped voltage to the waveform processor 1010 and the programmable current source 1008.

Waveform processor 1010 controls the programmable current source 1008 and controls the polarity switch 1012. The programmable current source 1008 includes a feedback loop with the waveform processor 1010. Waveform processor 1010 monitors the current source to in order to determine that sufficient current is being passed between electrodes connected to electrode ports 1014 and 1015.

Programmable current source 1008 receives control signals from the waveform processor 1010 and converts them into an appropriate output current waveform.

Polarity switch 1012 receives a control signal from the waveform processor 1010 and a current signal from programmable current source 1008. The polarity switch 1012 supplies the generated current waveform to electrode ports 1014 and 1015. In this embodiment polarity switch 1012 is a double pole change over switch utilising solid state analogue switches to switch polarity of the outputs. The direction of the current waveform applied between electrode ports 1014 and 1015 can thereby be switched.

The electrical generator circuit 1000 can be programmed to generate current waveforms with different parameters and can control the frequency and duration of application of current waveforms. This allows treatment of wounds to be tailored to individual patients needs. Furthermore, the battery allows electrical signals to be generated for over 35 hours. The system is therefore suitable for long-term treatments.

A further embodiment of a device for generating electrical waveforms is shown in FIG. 11B. In this embodiment the device 2000 comprises a polarity and connection sequence switch 2012, a waveform and sequence processor 2010, a voltage multiplier 2006, a programmable current source 2008, a power control processor 2004, a battery 2002 and a plurality of electrode ports 2014, 2015 and 2016. The device 2000 can be connected to electrode arrangements through electrode ports 2014, 2015 and 2016.

Power control processor 2004 operates in a similar fashion to power control processor 1004 of the previous embodiment.

The waveform and sequence processor 2010 controls the programmable current source 2008 and the polarity and connection sequence switch 2012. Programmable current source 2008 receives control signals from the waveform processor 2010 and converts them into an appropriate output current waveform.

Polarity and connection switch 2012 receives a control signal from the waveform and sequence processor 2010 and a current waveform signal from programmable current source 2008. The polarity and connector switch supplies the current waveform to a pair of appropriate electrode ports 2014 and 2015, 2014 and 2016, or 2015 and 2016 based on the control signals received from the waveform and sequence processor 2010. The current waveforms may also be applied to flow from two ports to one port or from one port to two ports simultaneously. The polarity and connection switch 2012 uses solid state analogue switches to switch the polarity of the current waveform and connect different combinations of electrode ports to the programmable current source. This arrangement allows the direction of the current to be switched and different combinations of electrode ports to be used.

Although in this embodiment the generator has only three electrode ports, any number of ports may be used.

Although in the embodiments of FIG. 11a and 11b, a cell battery, a power control processor and a voltage multiplier are used to power the circuit, in alternative embodiments a single battery cell of higher voltage may be used to replace the battery, the power control processor and the voltage multiplier.

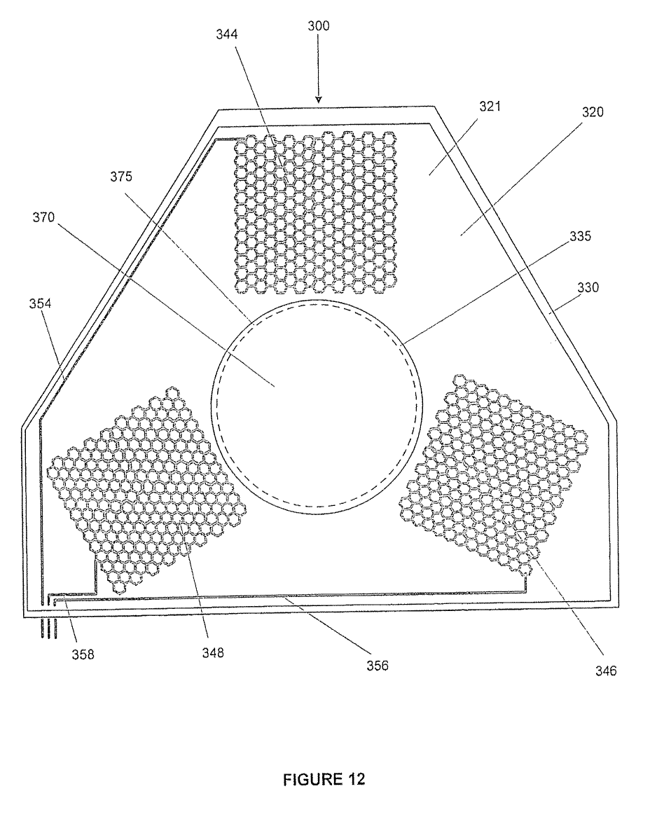

FIG. 12 is a schematic view of an electrode arrangement 300 according to a fifth embodiment of the invention. In this embodiment the electrodes are provided on one common printed circuit board. The electrode arrangement 300 comprises a common printed circuit board 320, three electrodes 344, 346 and 348 comprising a mesh structure of conductive tracks and conductive gel on a first surface 321 of the printed circuit board 320. Perforations 375 allow a portion of the substrate to be removed to create an aperture 370 located in the central region of the printed circuit board 320. The electrodes 344, 346 and 348 are arranged around the aperture 370. Each electrode is electrically isolated from another electrode. Connectors 354, 356, and 358 provide electrical signals to the electrodes 344, 346, and 348, respectively, when connected to an electrical generator. A strip of waterproof, electrically non-conductive sealing gel 330 is disposed around the edges of the printed circuit board 320 to prevent the ingress of moisture into the electrode arrangement 300. An annulus of electrically non-conducting gel 335 is disposed around the edges of the aperture 370. This arrangement has the advantage that the electrodes are provided on a common substrate. As well as being practical, since the electrodes are fixed on the substrate the relative position between the electrodes will not change during an electrotherapy procedure.

Although in this embodiment the substrate is provided with three electrodes, it may be appreciated that any number of electrodes may be provided on the surface to provide finer angular steps of electrodes around a wound.

Although in this embodiment electrically non-conductive sealing gel is placed around the edges of the aperture 370 and the edges of the printed circuit board, in alternative embodiments the sealing gel may be placed around each electrode provided on the substrate.

In alternative embodiments, the printed circuit board may already be provided with an aperture.

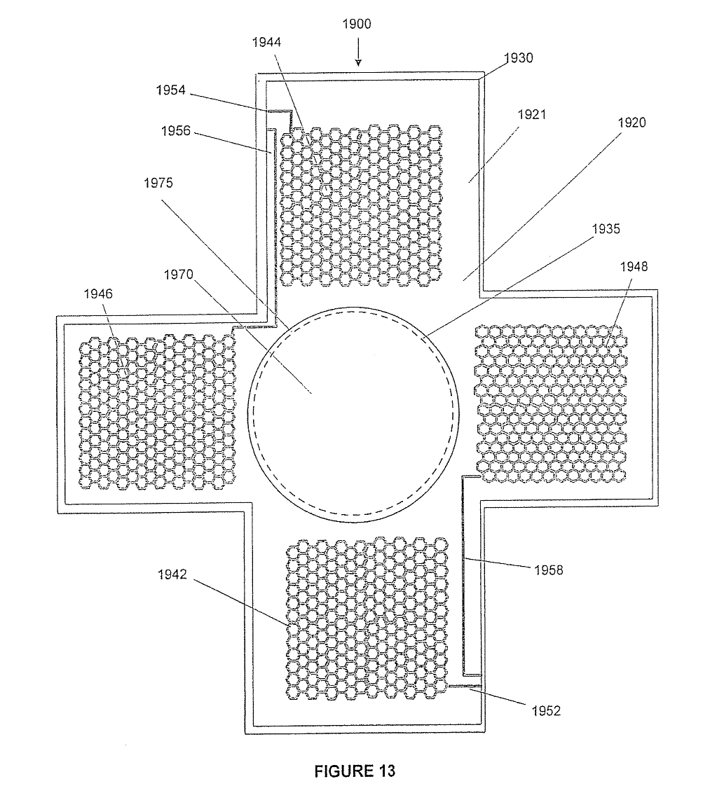

FIG. 13 is a schematic diagram of an electrode arrangement 1900 according to a sixth embodiment of the invention. In this embodiment four electrodes are provided on one common printed circuit board. The electrode arrangement 1900 comprises a common printed circuit board 1920, four electrodes 1942, 1944, 1946 and 1948 comprising a mesh structure of conductive tracks and conductive gel on a first surface 1921 of the printed circuit board 1920. Perforations 1975 allow a portion of the substrate to be removed to create an aperture 1970 located in the central region of the printed circuit board 1920. The electrodes 1942, 1944, 1946 and 1948 are arranged around the aperture 1970. Each electrode is electrically isolated from another electrodes. Connectors 1952, 1954, 1956 and 1958 provide electrical signals to the electrodes 1942, 1944, 1946 and 1948 respectively, when connected to an electrical generator. A strip of waterproof, electrically, non-conductive sealing gel 1930 is disposed around the edges of the printed circuit board 1920 to prevent the ingress of moisture into the electrode arrangement 1900. An annulus of electrically non-conducting gel 1935 is disposed around the edges of the aperture 1970. This arrangement has the advantage that the electrodes are provided on a common substrate.

Although in this embodiment the substrate is provided with four electrodes, it may be appreciated that any number of electrodes may be provided on the surface to provide finer angular steps of electrodes around a wound.

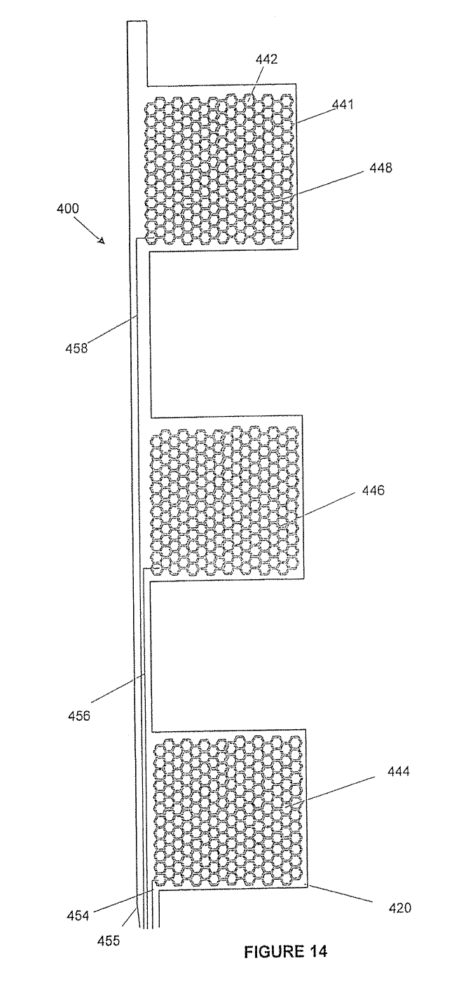

FIG. 14 is a schematic view of an electrode arrangement 400 according to a seventh embodiment of the invention. The electrode arrangement comprises a substrate 420, three electrodes 444, 446 and 448 provided on to first surface of the substrate 420. Each of the electrodes comprises electrically conductive tracks 441 and conductive gel 442. The substrate has an extended portion 455 carrying electrical connectors 454, 456 and 458. Electrical connectors 454, 456 and 458 are electrically isolated from one another. Electrical connector 454 is connected to electrode 444, electrical connector 456 is connected to electrode 446 and electrical connector 458 is electrically connected to electrode 448. The flexible substrate allows the electrodes 444, 446 and 448 to be bent into a shape that can be placed around a wound.

Again, although in this embodiment three electrodes are provided on the substrate any number of electrodes may be provided on the substrate.

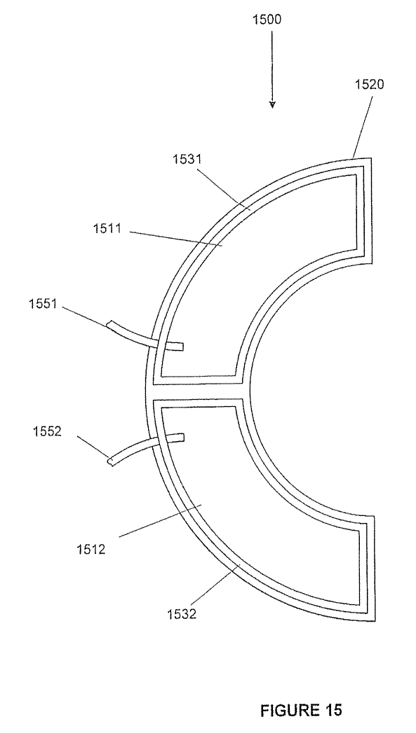

FIG. 15 is a schematic plan view of an electrode arrangement 1500 according to an eighth embodiment of the invention. The electrode arrangement 1500 comprises a substrate 1520 formed in a part annular shape, and having a first surface and a second surface opposed to the first surface. Two electrodes 1511 and 1512 are provided on the first surface of the substrate 1520. The electrodes 1511 and 1512 are each formed in a part annular shape and comprise electrically conductive tracks and electrically conductive gel. The electrodes are electrically insulated from one another. Connectors 1551 and 1552 provide electrode signals to the electrodes 1511 and 1512, respectively when connected to an electrical generator. A strip of waterproof, electrically non-conductive sealing gel 1531 is disposed around the edges of the electrode 1511 and over electrical connector 1551 to prevent ingress of moisture to the electrically conductive parts of the electrode 1511 and the electrical connector 1551. A strip of waterproof, electrically non-conductive sealing gel 1532 is disposed around the edges of the electrode 1512 and over electrical connector 1552 to prevent ingress of moisture to the electrically conductive parts of the electrode 1512 and the electrical connector 1552. This arrangement has the advantage that the electrode arrangement may be placed close to the edge of the wound.

While in this embodiment the substrate has a part annular shape, in alternative embodiments of the invention, the substrate may have a crescent shape. It should also be readily appreciated that while FIG. 15 shows a part annular substrate covering an angle of around 180 degrees, in alternative embodiments the part annular substrate may cover any angle from 0 to 360 degrees.

Although in this embodiment each electrode arrangement has two electrodes, in alternative embodiments each electrode arrangement may have one electrode or any number of electrodes.

Furthermore, although adhesive sealing gel is disposed around each electrode of the electrode arrangement in the above embodiment, in alternative embodiments the conductive gel may be disposed around the edges of the electrode arrangement and over each connector.

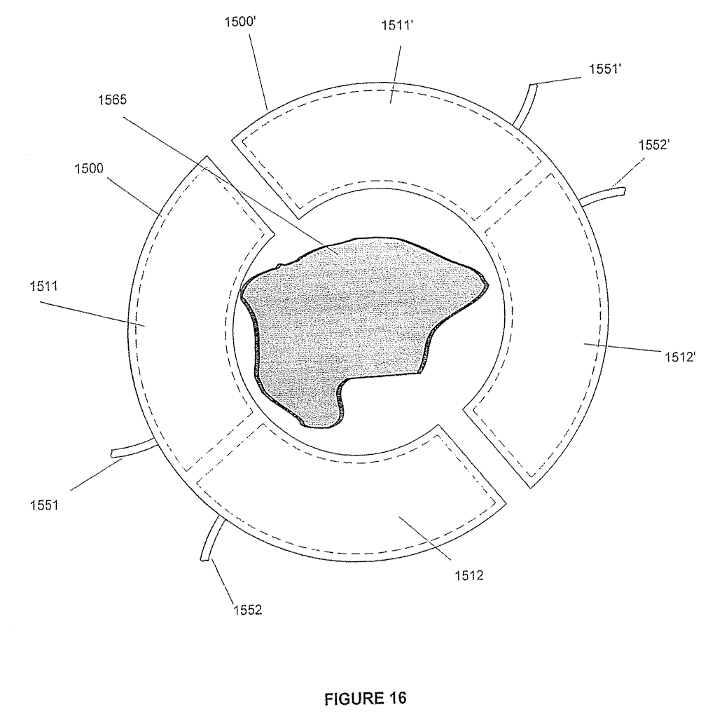

FIG. 16 is a schematic view of an area of treatment in which electrode arrangements according to the eighth embodiment of the invention are placed around a wound 1565 of a human patient. Electrode arrangements 1500 and 1500′ are placed on the skin on diagonally opposite sides of the wound 1565 with the first surface of the substrate of each electrode arrangement arranged to face the skin. Each electrode arrangement is pressed against the skin in order to adhere the adhesive sealing gel around each electrode 1511 and 1512, respectively, to the skin. This creates a seal preventing the ingress of moisture to any exposed conductive components of the electrode arrangements. Connectors 1551 and 1552 provide electrical signals to electrodes 1511 and 1512 respectively from an electrical generator (not shown) and connectors 1551′ and 1552′ provide electrical signals to electrodes 1511′ and 1512′. electrical current can therefore be applied between different electrodes passing through different paths through the regenerative tissue under the wound. For example electrical current may be applied to pass from electrode 1511 to electrode 1512′ and electrical current may pass from electrode 1512 to electrode 1511′. The current may be applied to flow in the opposite direction. Alternatively, electrical current may be applied to pass from electrode 1511 to electrode 1511′ and from electrode 1512 to electrode 1512′. The current may be applied to flow simultaneously in opposing directions under the wound. For example the current may be applied to flow from electrode 1511 to electrode 1512′ and from electrode 1511′ to electrode 1512.

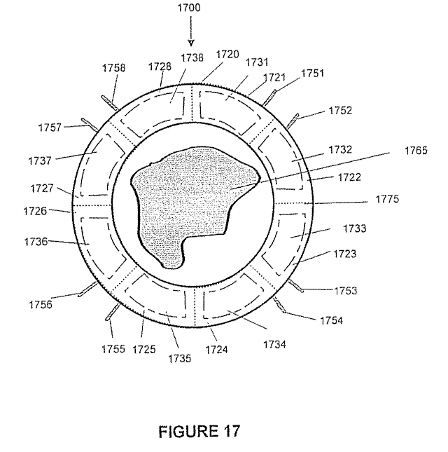

FIG. 17 is a schematic diagram of a second side of an electrode arrangement according to a ninth embodiment of the invention. The electrode arrangement is placed in an area of treatment around a wound 1765. The electrode arrangement 1700 comprises a substrate 1720 in the form of an annulus. The substrate is subdivided along the radial axes into 8 substantially equally sized portions 1721, 1722, 1723, 1724, 1725, 1726, 1727 and 1728 positioned around the aperture of the annulus. Each portion has a conductive area on a first surface of the substrate opposed to the second surface, these are shown as dotted areas 1731, 1732, 1733, 1734, 1735, 1736, 1737 and 1738, respectively. Each conductive area is made up of mesh structure of conductive tracks and a conductive gel. Perforations 1775 disposed radially between adjacent portions allow one or more of portions 1721, 1722, 1723, 1724, 1725, 1726, 1727 and 1728 to be removed. This arrangement allows portions of the substrate to be removed to facilitate treatment of different size and shapes of wounds. Connectors 1751, 1752, 1753, 1754, 1755, 1756, 1757, 1758 provide electrical signals to electrodes 1721, 1722, 1723, 1724, 1725, 1726, 1727 and 1728, respectively, when connected to an electrical generator. Although the common substrate in this embodiment is in the form of a ring, the common substrate may in the form of any shape with an aperture in the central region of the substrate.

It may be appreciated that while in this embodiment the common substrate completely surrounds the wound, in alternative embodiments the common substrate may be part annular and partially surround the wound.

Although in this embodiment the substrate is divided into 8 portions it may be understood that the substrate may be subdivided into any number of portions. Furthermore, although perforations are provided along the borders between adjacent portions in this embodiment, in alternative embodiments perforations may not be present and the substrate may be cut or broken along a radial line to remove portions.

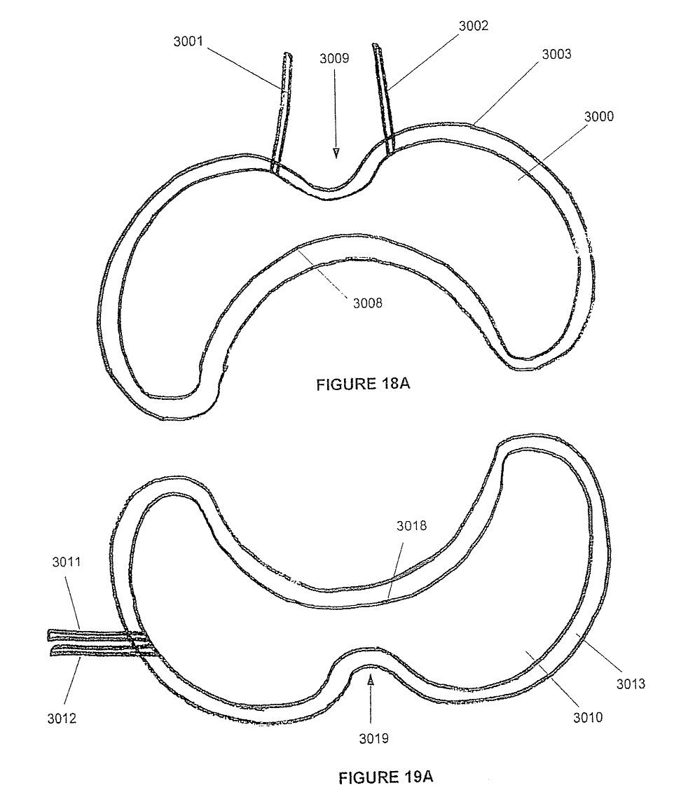

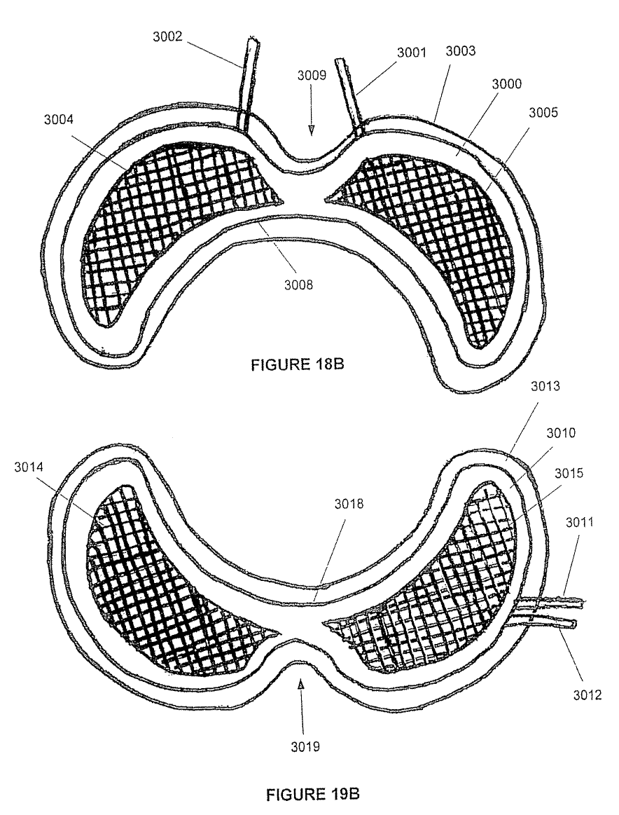

FIGS. 18A and 18B are diagrams illustrating an electrode design according to a tenth embodiment of the present invention. In this design the electrode is shaped as two linked crescent shaped electrode regions lying in a generally part annular shape. The generally crescent shaped regions are linked at their apices. The electrode is formed of a non-conductive substrate 3000 which can be waterproof to provide a sealed electrode. The substrate 3000 is shaped as two crescent shaped portions linked at their ends to form a part annular shape having a concave circumferential inner edge 3008 and a circumferential outer edge shaped to form a recess 3009. On a first surface (generally a ‘bottom’ surface which will lie towards the skin of the patient) of each crescent shaped region of the substrate 3000 electrically conductive electrode regions 3004 and 3005 are provided. The electrode regions 3004 and 3005 are formed as layers of conductive material on the substrate 3000 in a similar manner to previous embodiments. The substrate 3000 and electrode regions 3004 and 3005 can comprise a flexible printed circuit board. The electrode regions 3004 and 3005 can be formed as a mesh or matrix of conductive material, a solid layer of conductive material, or a combination of a lower conductivity solid layer plus a layer of higher conductivity mesh or a matrix on either side of the solid lower conductivity layer. On a first surface of each electrode region 3004 and 3005 lying adjacent to the substrate 3000, respective electrical connector leads 3001 and 3002 electrically connect to the electrode regions 3004 and 3005 and lie between the substrate 3000 and the electrode regions 3004 and 3005.

The substrate 3000 extends beyond the electrode regions to form a flange region which is for adherence to the skin of the patient. Thus at least the flange regions of the substrate have an adhesive material applied thereto which is preferably waterproof to form a seal around the electrode regions 3004 and 3005 when the substrate 3000 is adhered to the skin of the patient. The substrate 3000 can also have an adhesive applied to the first surface in the region of the electrode regions 3004 and 3005 in order to bond the substrate to the electrode regions 3004 and 3005 and to bond the electrical connector leads 3001 and 3002 to the substrate 300 and the electrode regions 3004 and 3005 and to secure the electrical connection between the electrode regions 3004 and 3005 and the electrical connector leads 3001 and 3002.

The electrical regions 3004 and 3005 is provided with an electrically conductive gel on a second face away from the substrate for providing an electrical path to the skin of the patient from the electrode regions 3004 and 3005. The gel can be adhesive in nature to assist in the adherence of the electrode to the skin of the patient.

A release layer 3003 is provided across the electrode on the adhesive flange region of the substrate 3000 and on the adhesive second side of the electrode regions 3004 and 3005 to allow for storage of the electrode. The release layer 3003 is removed in order to adhere the electrode to the skin of the patient.

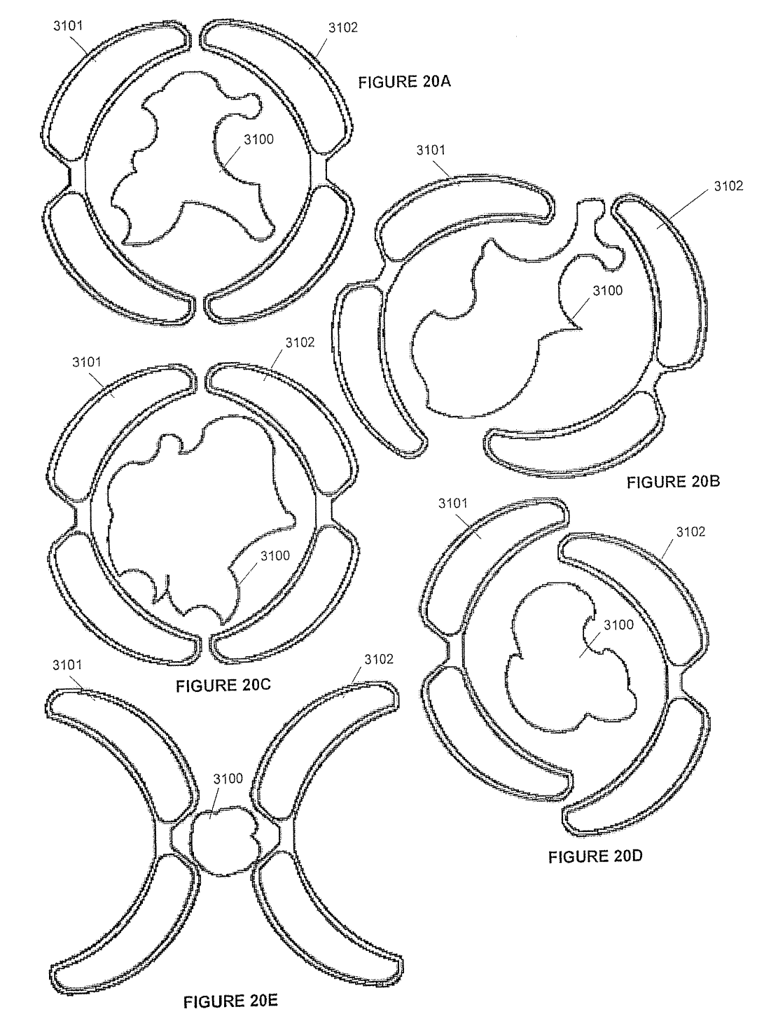

In this embodiment the electrical connector leads 3001 and 3002 are illustrated as being connected to and leaving the substrate in parallel either side of the recess in the outer circumferential edge of the substrate. While this provides for a symmetrical structure, the electrical connector leads 3001 and 3002 can get in the way of application of two of the electrodes around a small wound as will be described with reference to FIG. 20E.

FIGS. 19A and 19B illustrate the top and bottom views of an electrode according to an eleventh embodiment of the present invention. This design of electrode is identical to the design of the tenth embodiment of the invention except for the placement of the electrical connector leads 3011 and 3012. In this design the leads 3011 and 3012 exit from the substrate 3010 at a region remote from the recess 3019. This ensures that the leads 3011 and 3012 will not lie in the region of a small wound as illustrated in FIG. 20E. In order for the leads of a pair of electrodes placed either side of a wound to lie in parallel, a pair of electrodes can be provided having leads exiting from the substrate 3010 at opposite crescent shaped regions i.e. they can be mirror image designs.

FIGS. 20A to E illustrate placement of the electrodes of the tenth or eleventh embodiment of the invention around different size and shapes of wounds. In FIGS. 20A to D it can be seen that the electrodes 3101 and 3102 are arranged with their inner circumferential edges facing the wound 3100. They can be arranged in slightly different positions to get close to the wound 3100. In FIG. 20E it can be seen that the wound 3100 is small and hence the inner circumferential edge of the electrodes is too large a diameter to enable the electrodes to be positioned close to the wound. Hence the electrodes are placed with the recessed portion of their outer circumferential edge adjacent to the wound. In this way the electrode regions are in close proximity to the wound to provide electrical stimulation therapy for wound healing.

It can thus be seen that the joined crescent shaped electrodes enable closer placement of the electrode regions to wounds with a variety of shapes and sizes. Current can be passed between any of the four electrode regions or between diagonally opposed pairs only.

The leads in the tenth and eleventh embodiments of the invention can be formed as flat leads in the same manner as the third embodiment of the invention, thus making the electrode suitable for use under a compression dressing.

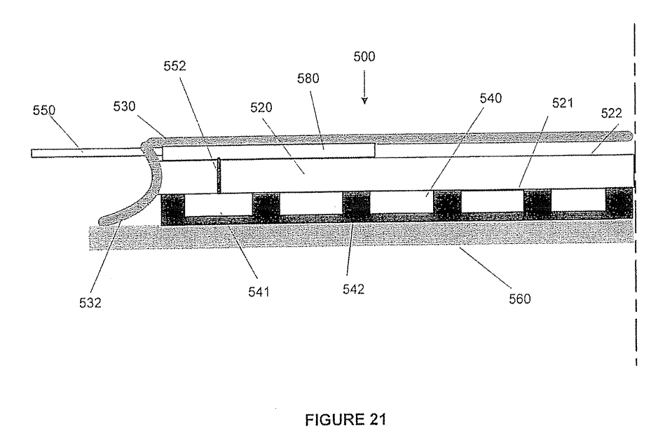

FIG. 21 is a partial cross-sectional view of an electrode arrangement 500 according to a twelfth embodiment of the invention. The electrode arrangement 500 comprises a printed circuit board 520 having a first surface 521 and a second surface 522 opposed to the first surface, electrically non-conductive waterproof sealing pad 530, an electrode 540 for applying electrical signals to the skin 560 when placed in contact with the skin and an electrical generator circuit 580. The electrode 540 is provided on the first surface of the printed circuit board 520 and comprises electrically conductive tracks 541 and electrically conductive gel 542 to provide electrical conductivity across a portion of the surface of the printed circuit board. The electrical generator circuit 580 is placed on the second surface of the printed circuit board 520 and connector 552 provides an electrically conductive connection between the electrical generator circuit 580 and electrode 540 through the printed circuit board 520. Electrical lead 550 can be used to connect the electrode arrangement to another electrode arrangement. The sealing pad 530 is placed over the second surface of the printed circuit board 520. The outer edges 532 on one surface of the sealing pad 530 have adhesive properties and the sealing pad 530 is positioned such that the edges 532 of the sealing pad extend beyond the edges of the printed circuit board 520 and adhere the electrode arrangement to the skin. Such an arrangement prevents ingress of moisture such as sweat, urine, blood or wound exudate to the electrode 540 when the electrode arrangement 500 is placed on the skin and to the electrical generator circuit 580 and the electrical connection between the electrical generator circuit and the electrical lead 550. The electrical generator 580 contains the circuitry shown in FIG. 11A and has a surface area small enough to fit on the back of the printed circuit board. The thickness of the electrical generator 580 is approximately 6 mm. The small size of the electrical generator allows it to be easily integrated onto the printed circuit board providing a small and compact electrode arrangement.

While in this embodiment the generator is described as having a thickness of approximately 6 mm, it may be appreciated that the electrical generator may be of any size that fits on the back of the electrode and that can be carried comfortably by the patient.

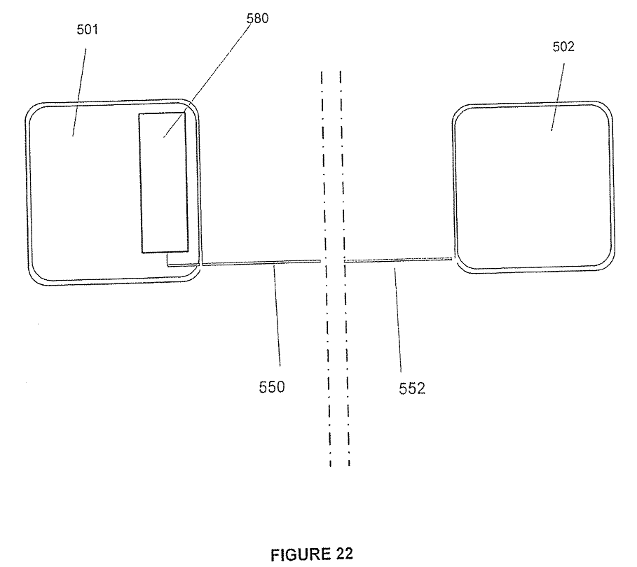

FIG. 22 is a schematic view of the embodiment of the electrode arrangement 501 shown in FIG. 21 connected to another electrode arrangement 502. Current from electrical generator 580 is applied across electrode arrangements 501 and 502 through connectors 550 and 552.

It may be appreciated that although in this embodiment electrical generator 580 is connected to one other electrode arrangement it may be connected to any number of electrode arrangements to apply current between electrode arrangement 501 and any number of electrode arrangements.

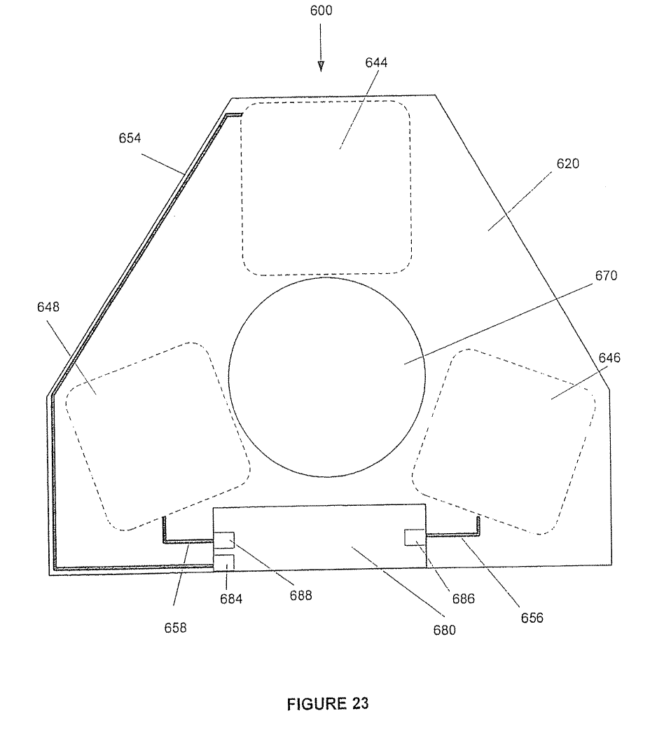

FIG. 23 is a schematic view of a second side of an electrode arrangement according to a thirteenth embodiment of the invention. The electrode arrangement 600 comprises a common printed circuit board 620 and three electrodes provided on the first surface of the printed circuit board 620 which are shown in the figure as dotted areas 644, 646 and 648. An aperture 670 is located in the central region of the printed circuit board 620. The electrodes 644, 646 and 648 are arranged around the aperture 670. Each electrode is electrically isolated from another electrode. An electrical generator circuit 680 is provided on the second surface of the electrode arrangement 600. Connectors 654, 656, and 658 provide electrical signals from output ports 684, 686, and 688 of the electrical generator circuit 680 to the electrodes 644, 646, and 648, respectively. An annulus of electrically non-conductive sealing gel is disposed around the edges of the aperture 670 on the first surface 621 of the electrode arrangement to prevent the ingress of moisture into the electrode arrangement 600. An electrically insulating waterproof adhesive material (not shown in the Figure) is placed over the second surface of the electrode arrangement to cover the substrate 620 and the generator 680 sealing the electrode arrangement 600 to skin when placed on the skin and preventing the ingress of moisture to any exposed components. This arrangement has the advantage that the electrode arrangement is completely self contained and does not need to be connected to an external electrical generator circuit. Such an arrangement may be disposable after use.

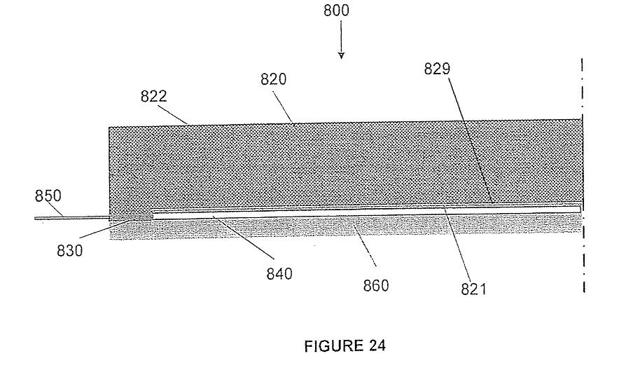

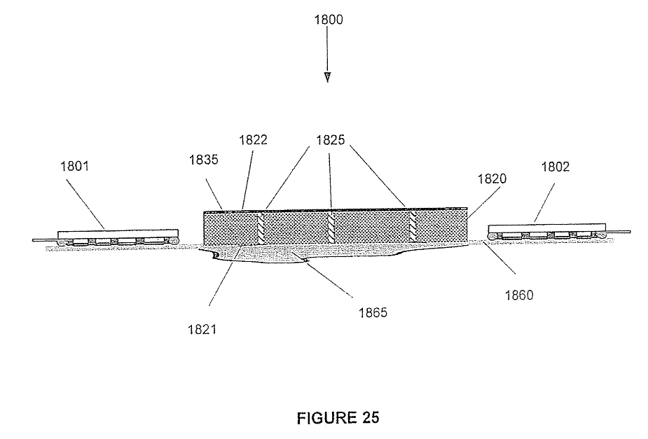

FIG. 24 is a partial cross sectional view of an electrode arrangement 800 placed on the skin 860 of a patient according to a fourteenth embodiment of the present invention. The electrode arrangement 800 comprises a porous layer of foam 820 having a first surface 821 and a second surface 822 opposed to the first surface, an electrode 840 for applying electrical signals to the skin 860 when placed in contact with the skin and an electrically conductive lead 850 for supplying electrical signals to the electrode 840. A layer of electrically insulating, waterproof, adhesive material 829 is disposed on the first surface of the foam layer and adheres the electrode 840 to the foam layer 820 and prevents the ingress of moisture from the foam layer 820 to the electrode 840. Waterproof, electrical insulating, adhesive material is disposed on the outermost edges of the first surface 821 and around the electrically conductive lead 850, forming a seal 830 adhering the electrode arrangement to the skin and preventing the ingress of moisture to any exposed conductive elements of the electrode arrangement. Such an arrangement provides an electrode arrangement which is easy to apply and an overall structure which remains effective for longer. In addition, the layer of foam absorbs excess fluids from outside the electrode arrangement.

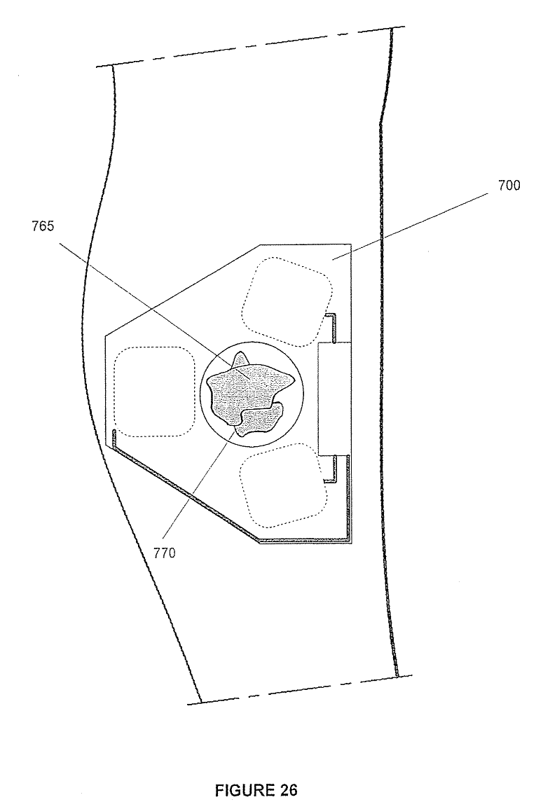

FIG. 25 is a partial cross sectional view of a pad for use with an electrode arrangement comprising at least two electrodes for applying electrical signals to the skin of an animal. The pad 1800 comprises a layer of absorbent foam 1820 having a first surface 1821 and a second surface 1822 opposed to the first surface. A plurality of strips 1825 is arranged substantially parallel to one another across the layer of foam 1820. The strips extend from the second surface 1822 of the layer of foam 1820 to the first surface 1821 and are made of an electrically insulating material. A layer of electrically insulating, adhesive material 1835 is disposed on the second surface of the foam layer 1820. The pad 1800 is placed over a wound 1865. An electrode 1801 for applying electrical signals to the skin is placed in contact with the skin on side of the device 1800, and another electrode a 1802 is placed in contact with the skin on the other side of the pad opposite the first electrode 1801. The layer of foam 1820 provides an absorbent medium for absorbing fluid from the wound 1865. The strips of insulating material 1825 arranged perpendicularly to the path of current flow between the two electrodes provide a barrier to any electrical conductive path which may be created through the layer of foam 1820 by the presence of the exuded fluid in the foam. Such an arrangement helps to eliminate any low resistance path which may be created from the exuded fluid and which would otherwise short circuit current that should be passing through the regenerative tissues under the wound.

In an alternative embodiment of the invention the pad may consist of a plurality of trenches filled with an electrically insulating material.

In further embodiments of the pad, the strips of insulating material or the trenches filled with electrically insulating material may be arranged in a honeycomb grid structure across the layer of foam. In this embodiment the pad may be used with three or more electrodes arranged around a wound and a current path in any direction through the foam may be prevented.

An embodiment of such an electrode arrangement is shown in FIG. 25. The electrode arrangement comprises electrodes 2801, 2802, 2803 and 2804 arranged around a wound and a foam pad 2820 placed over the wound. The foam pad comprises a plurality of interconnected electrically insulating portions 2825 arranged in a honeycomb pattern. The insulating portions 2825 provide a barrier to any electrical conductive path of current flow between any of the electrodes 2821, 2822, 2823 and 2824 through the foam pad 2820 which may be created from exuded fluid in the foam.

It will be understood that the absorbent material and electrically insulating portions may be distributed in any regular or irregular pattern in 1D or 2D achieving the function of providing a resistance to current flow in one or any direction through the foam.

It will also be appreciated that any form of absorbent material may be used in place of foam.

FIG. 26 is a schematic view of an area of treatment on the leg of a patient in which an electrode arrangement 700 of FIG. 23 is placed around a wound 765 such that the aperture 770 is placed over the wound 765.

In electrotherapy, when electrodes are place on a patients skin a current passes between the electrodes and a concentration of current can occur at the edges. There are a number of causes. The shortest distance between the electrodes is between facing edges and the boundary near the edge. Exudate or moisture can “short circuit” the edge of the gel between conductor and skin bridging out the gel. There can be distortion of the gel. It is advantageous to reduce these effects.

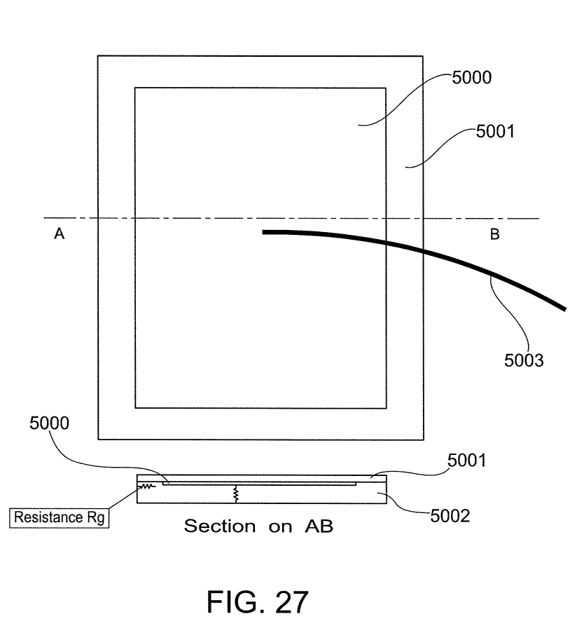

FIG. 27 illustrates an embodiment of the present invention in which a conductive layer 5000 is printed on a non-conductive substrate 5001. A conductive gel 5002 is provided to provide conduction to the skin of a patient. The conductive area that is in contact with the gel finishes short of the edge of the gel 5002. This boundary edge exhibits resistance to the edge (in a lateral direction) which is several times that of the back to front (perpendicular direction) resistance of the gel in view of the much larger length of the gel in the lateral direction compared to the thickness of the gel in the perpendicular direction (the thickness of the gel 5002 in FIG. 27 is shown greatly expanded for ease of illustration only). Thus by providing a boundary of gel around the conductive area of the electrode the effects of edge burning can be reduced since current will preferentially take the path of least resistance from the electrode perpendicularly to the skin.

In this embodiment the edge thickness of the gel provides a sealing arrangement between the substrate 5001 and the skin which is of high electrical resistance. Hence both electrical and hermetic isolation is provided for (assuming the substrate is waterproof). However, any sealing arrangement of a previous embodiment can also be employed. Further, the principle of providing a boundary area of conductive gel around the edge of the conductor (inside any peripheral sealing arrangement) can be used with any previously described embodiment.

Although in the above embodiments, the electrically conductive tracks or elements are etched out of gold plated copper, it may be appreciated that they may be etched, printed or otherwise fabricated out of any electrically conductive material. Furthermore, it may be appreciated that the electrically conductive tracks are etched in the form of a honey comb mesh pattern, it may be appreciated that any mesh or grid pattern or other any pattern of tracks allowing flexibility of the electrode arrangement may be used. It may also be appreciated that any type of flexible substrate may be used in place of a printed circuit board.

Although in FIG. 1, electrically non-conductive sealing gel placed around the edges of the printed circuit board is used to seal the electrode arrangement to the skin, in other embodiments a waterproof adhesive material may be placed over the second surface of the printed surface board to adhere the electrode arrangement to the skin.

In alternative embodiments, the electrode arrangement of FIG. 1 may include an electrical generator on the second surface of the printed circuit board. The electrical generator may be sealed by covering it with a waterproof sealing pad or alternatively the generator may be contained within a sealed unit.

Although in the embodiment of FIG. 4, the electrically conductive elements are square shaped, it should be appreciated that they may have any geometrical shape.

Although in the embodiment of FIG. 5 electrically non-conductive sealing gel placed around the edges of the printed circuit board is used to seal the electrode arrangement to the skin, in other embodiments a waterproof adhesive material may be placed over the second surface of the printed surface board to adhere the electrode arrangement to the skin.

In alternative embodiments, the electrode arrangement of FIG. 5 may include an electrical generator on the second surface of the printed circuit board. The electrical generator may be sealed by covering it with a waterproof sealing pad or alternatively it may be contained within a sealed unit.

It may be appreciated that the different embodiments of electrode arrangements may be connected to any type of external electrical generator circuit providing current.

Although in FIG. 10, connectors 251, 252 and 253 protrude from the top of the bandage for connection to the electrical generator, in alternative embodiments the connectors may protrude from the bandage at any level of the bandage. Alternatively the treatment area may not be bandaged. In further embodiments, the electrical generator may not be attached to the leg.

Although in FIG. 21, electrical connection between the electrode and the electrical generator is made by a connector through the printed circuit board, in alternative embodiments electrical connection may be made between the electrode and the electrical generator through a connector passing by the exterior of the printed circuit board.

Although the embodiment of FIG. 24 has only one electrode, in alternative embodiments, two or more electrodes may be provided on the foam layer.

The electrode arrangement can include a flexible electrically non-conductive substrate, one or more electrodes provided on a first surface of the substrate for applying electrical signals to the skin when placed on the skin, one or more connectors each connected to an individual electrode for providing electrical signals to the respective individual electrode, and an electrically non-conductive sealing arrangement connected to the substrate for sealing the substrate and the skin to prevent ingress of moisture to the at least one electrode and to the at least one connector at the connection to the respective electrode. Thus in accordance with this aspect of the invention, the electrode arrangement is resistant to the ingress of moisture such as urine, perspiration and blood which may lead to electrical short circuits and disruption to the functioning of the electrode system. This is particularly beneficial when using the electrode to perform electrotherapy on patients who may be incontinent or for use in wound healing where the wounds seep plasma or exude infected material.

The electrically non-conductive sealing arrangement can be applied to an edge region of the substrate and disposed around said at least one connector. In another embodiment, the sealing arrangement comprises adhesive material for adhering the substrate to the skin. In a further embodiment, the first surface of the substrate includes a peripheral region where there is no electrode provided and the adhesive material of the sealing arrangement is applied to this peripheral region. In another embodiment, the sealing arrangement is placed over a second surface of the substrate opposed to said first surface.

Electrical components can be provided on the second surface of the substrate. This arrangement allows electrical connections to be made to the or each electrode on the side of the electrode arrangement not in contact with the skin.

Each electrode can comprise a plurality of interconnected electrically conductive elements to allow flexion of the electrode arrangement. Such an arrangement allows effective electrical coupling to be made between an electrode and the skin even when the surface to which the electrode is being applied is uneven or curvaceous.

The plurality of interconnected electrically conductive elements can be arranged in a mesh pattern to allow flexion of the electrode arrangement.

The plurality of interconnected electrically conductive elements can be arranged in a matrix.

The electrode arrangement can comprise electrically conductive gel on the electrode. This arrangement enhances the electrical conductivity across the surface of the electrode and provides further effective electrical coupling between the electrode and the skin.

The substrate can have a part annular shape. Such an arrangement allows the edge of the electrode arrangement to be placed close to the edges of a wound.

The substrate can have an annular shape. This arrangement allows the electrode arrangement to be placed on the skin of a patient surrounding a wound.

The substrate can be porous. This arrangement provides an electrode arrangement which is easy to apply and an overall structure which remains effective for longer periods of time.

One or more elements of highly electrical resistant material can be provided on a surface of each electrode. This arrangement provides a more even distribution of electrical current over the surface of the electrode.

The elements of highly resistive material can be interspaced by elements of electrically insulating material thereby allowing isolated pockets of high resistance material to be created on the surface of the electrode.

A device for use with the electrode arrangement according to any embodiment of the invention for absorbing fluid from a wound can comprise one or more elements of absorbent material and a plurality of interconnected portions of electrically insulating material interposed between the elements of absorbent material.

An electrode arrangement for applying electrical signals to skin of an animal can includes a flexible electrically non-conductive substrate and at least one electrode provided on a first surface of the substrate for applying electrical signals to the skin when placed on the skin, wherein the substrate includes an extended portion comprising at least one electrically conductive region, the or each electrically conductive regions being in electrical contact with a respective electrode to form a connector to said electrode. Thus in accordance with this aspect of the invention, an electrode arrangement with a low profile connecting lead is provided. The connector being substantially flat will not indent the skin of the patient and help to minimise any discomfort. Furthermore, such an arrangement will not result in raised areas and bulges in bandaging which may be applied over the electrode arrangement during treatment.

An electrode arrangement for applying electrical signals to skin of an animal can comprise a flexible electrically non-conductive substrate, at least one electrode on a first surface of the substrate for applying electric signals to the skin when placed on the skin, and an electrical generator circuit on a second surface of the said substrate opposed to the first surface of said substrate to apply electrical signals to said at least one electrode. This provides an electrode arrangement which is self contained and can operate autonomously without the need to be connected to an external electrical current generator in order to function. The electrode arrangement can therefore be used without using cumbersome external leads. Furthermore, the patient undergoing electrotherapy can move around without having to move a separate electrical generator device around with him. Treatment can thereby be practically implemented over long periods of time without intervention and electrotherapy can be performed on a patient in the comfort of his own home.

The electric generator circuit can be adapted to switch current to flow between different electrodes of the at least three electrodes. This arrangement allows current to be applied through different paths across an injured area of tissue thereby enhancing electrotherapy treatment techniques

The electrical current generator can be adapted to switch the direction of current flow between electrodes. This arrangement allows different current profiles to be applied across an area of treatment.

The electrical current generator can comprise a waveform generator for generating current waveforms across said electrodes. This allows different current waveforms to be applied across the area of treatment and the treatment to be adapted to the patient.

The waveform generator can be pre-programmed with at least one program to generate a pre-determined waveform or a pre-determined sequence of predetermined waveforms. Standard treatment programs can therefore be programmed into the system and automatically run, resulting in a user-friendly system.

An apparatus for treating a wound can comprise a flexible electrically non-conductive substrate, at least three electrodes on a first surface of the substrate for arrangement around the wound, at least three connectors, each connected to a respective electrode adapted to allow current to flow to each of the at least three electrodes when connected to a current generator and an electrical current generator for applying current to said connectors and to switch current between pairs of electrodes. This aspect of the invention provides an apparatus for treating wound in which the electrical current can pass through different paths through the tissue under the wound thereby providing more effective treatment of the wound. Furthermore, since the electrodes are fixed on a common substrate relative position between electrodes does not change during treatment.

A method for treating a wound can involve placing at least three electrodes on the skin around the wound, applying electrical current between electrodes of the at least three electrodes and switching the current to flow between different electrodes of the at least three electrodes. This provides a new and improved method whereby electrical current can take different paths across the wound simultaneously or in cycles resulting in an increase in the efficacy of wound healing. Any type of wound such as venous ulcers, pressure sores, diabetic ulcers may be treated using such a method.

A pad for application to a wound to extend laterally across the wound can comprise one or more elements of absorbent material for absorbing fluid exuded from the wound and distributed in a pattern laterally across the pad; and a plurality of portions of electrically insulating material interposed laterally between the elements of absorbent material to provide high resistance to current flow in at least one direction laterally across the pad.

Such an arrangement helps to eliminate any low resistance path which may be created from exuded fluid from a wound absorbed by the pad and which would otherwise short circuit current that should be passing through the regenerative tissues under the wound when electrotherapy treatment is being carried out with electrodes placed around the wound.

An electrode arrangement for applying electrical signals to the skin of an animal can comprise an electrically non-conductive substrate at least one electrode provided on a first surface of the substrate for applying electrical signals to the skin when placed on the skin wherein a high resistance is connected in series with the electrode.

Such an arrangement results in a high resistive path being created and renders any resistance of the skin and the electrode negligible in comparison to the high resistance. Consequently, any differences in resistances of the electrode and the skin across the surface of the electrode resulting from uneven contact or skin non-linearity will be rendered imperceptible. The result will be a more even distribution of current across the surface of the electrode.

The present invention has applicability for any animal but can be particularly used on humans.

Although the present invention has been described with reference to specific embodiments, it will be apparent to a skilled person in the art that modifications lie within the spirit and scope of the present invention. In particular, the different features from different embodiments may be interchanged, where appropriate.

What is claimed is:

1. An electrode arrangement for applying electrical signals to skin of an animal comprising:
a flexible substrate;
at least one electrode provided on a first surface of the substrate for applying electrical signals to the skin when placed on the skin;
electrically conductive gel applied to said at least one electrode for providing a conductive path from said at least one electrode to the skin;
at least one connector lead for providing electrical signals to the respective electrode and having an electrically conductive part enclosed within a non-conductive covering; and
an electrically non-conductive sealing arrangement connected to said substrate for preventing the ingress of moisture to said at least one electrode, said electrically conductive part of said at least one connector at the connection to said at least one electrode, and said electrically conductive gel.

2. An electrode arrangement according to claim 1, wherein said electrically non-conductive sealing arrangement comprises a flexible sheet applied over a second surface of said substrate and comprising a first part covering said substrate and a second part extending laterally beyond said substrate, and an adhesive material applied to said second part of said flexible sheet for adhering said second part to said skin.

3. An electrode arrangement according to claim 2, wherein said at least one connector lead is arranged to extend from said substrate across said adhesive material so that a seal is formed around said at least one connector lead when said second part is adhered to said skin.

4. An electrode arrangement according to claim 1, wherein said electrically non-conductive sealing arrangement comprises said substrate covering said at least one electrode, at least one portion of said substrate extending beyond said at least one electrode, and an adhesive material applied to said at least one portion of said substrate for adhering to said at least one portion of said substrate to said skin.

5. An electrode arrangement according to claim 4, wherein said at least one connector lead is arranged to extend across said adhesive material so that a seal is formed around said at least one connector lead when said at least one portion of said substrate is adhered to said skin.

6. An electrode arrangement according to claim 4, wherein said at least one portion of said substrate extending beyond said at least one electrode has a width of 4 to 8 mm.

7. An electrode according to claim 1, wherein said electrically non-conductive sealing arrangement is arranged to effectively seal under a pressure of up to 50-60 mm Hg constant pressure for up to 7 to 9 days.

8. An electrode arrangement for applying electrical signals to skin of an animal comprising:
at least one flexible electrode for applying electrical signals to the skin when placed on the skin;
electrically conductive gel applied to said at least one electrode for providing a conductive path from said at least one electrode to the skin;
at least one connector lead connected to a respective said at least one electrode for providing electrical signals to the respective electrode and having an electrically conductive part covered by a non-conductive covering; and
an electrically non-conductive sealing arrangement covering said at least one electrode for preventing the ingress of moisture to said at least one electrode, said electrically conductive part of said at least one connector at the connection to said at least one electrode, and said electrically conductive gel.

9. An electrode according to claim 8, wherein said electrically non-conductive sealing arrangement is arranged to effectively seal under a pressure of up to 60 mm Hg constant pressure for up to 7 to 9 days.

10. A wound treatment arrangement for treating a wound in the skin of a patient, the wound treating arrangement comprising:
a compression dressing arrangement for application to the limb of the patient to apply pressure thereto; and
a flat electrode arrangement for applying electrical signals to said skin, said electrode arrangement comprising: a flexible electrically non-conductive substrate; and
at least one electrode provided on a first region of a first surface of said substrate for applying electrical signals to the skin;
wherein said at least one electrode is adapted for arrangement on said skin adjacent said wound under said compression dressing arrangement, said substrate includes an extended portion comprising at least one electrically conductive region, the or each electrically conductive region is in electrical contact with a respective electrode to form a respective connector to said electrode, and the at least one connector is adapted to extend under and out from said compression dressing arrangement to enable electrical signals to be applied to said skin.

11. A wound treatment arrangement according to claim 10, including an electrical generator apparatus for attachment to said compression dressing arrangement and connection to said at least one connector for generating electrical signals to be applied to said skin.

12. A wound treatment arrangement according to claim 10, wherein said at least one electrode comprises a plurality of interconnected electrically conductive elements to form a two-dimensional electrically conductive path across at least a portion of the first region of the first surface of the substrate.

13. A wound treatment arrangement according to claim 12, wherein the plurality of interconnected electrically conductive elements are arranged in a matrix to allow flexion of the electrode arrangement

14. A wound treatment arrangement according to claim 12, wherein the plurality of interconnected electrically conductive elements are arranged in a mesh pattern to allow flexion of the electrode arrangement

15. A wound treatment arrangement according to claim 10, wherein electrically conductive gel is provided on said at least one electrode to provide an electrically conductive path to the skin.

16. A wound treatment arrangement according to claim 11, wherein said at least one electrode comprises at least three electrodes and said at least one connectors comprises at least three respective connectors, wherein the electrical generator apparatus is adapted to switch current to flow between different electrodes of the at least three electrodes.

17. A wound treatment arrangement according to claim 10, wherein said first region of said substrate has a part annular or crescent shape.

18. A pad for application to a wound to extend laterally across the wound, the pad comprising:
one or more elements of absorbent material for absorbing fluid exuded from the wound and distributed in a pattern laterally across the pad; and
a plurality of portions of electrically insulating material interposed laterally between the elements of absorbent material to provide high resistance to current flow in at least one direction laterally across the pad.

19. A pad according to claim 18, wherein the portions of electrically insulating material are arranged in substantially parallel strips to provide high resistance laterally in at least one direction across the pad.

20. A pad according to claim 17, wherein the portions of electrically insulating material are arranged in a matrix to provide high resistance in any direction across the pad.

21. An electrode for applying current to the skin of a patient comprising:
a flat substrate having one of a substantially part annular shape and a substantially crescent shape;
at least one electrode region applied to one surface of the substrate;
at least one electrical connector connected to said at least one respective electrode; and
an adhesive material for adhering the electrode to the skin of the patient.

22. An electrode for applying current to the skin of a patient comprising:
a flat substrate shaped as two substantially crescent shaped portions apically linked and having a concave inner circumferential edge and a convex outer circumferential edge, said outer circumferential edge having an indented region situated at the apically linked region between said substantially crescent shaped portions;
at least one electrode region applied to one surface of the substrate;
at least one electrical connector connected to said at least one respective electrode region; and
an adhesive material for adhering the electrode to the skin of the patient.

23. An electrode according to claim 22, wherein said substrate and said adhesive material forms a seal to seal the electrode against the ingress of moisture to a conductive path from said at least one electrical connector to the skin.

24. An electrode according to claim 21, wherein said adhesive is arranged at a peripheral region of said substrate to provide a waterproof seal around said at least one electrode region, and said substrate is waterproof.

25. An electrode according to claim 21, including a conductive gel arranged on said at least one electrode region for conducting current between said at least one electrode region and the skin.

26. A method of treating a wound in the skin of a patient comprising
placing a plurality of electrodes having one of a substantially part annular shape and a substantially crescent shape on the skin around the periphery of the wound so as to at least partly encircle the wound; and
applying electrical current across the wound between said electrodes.

27. A method according to claim 26, wherein said electrodes comprise a substrate shaped as two substantially crescent shaped portions apically linked and having a concave inner circumferential edge and a convex outer circumferential edge, each substantially crescent shaped portion having a substantially crescent shaped electrode portion applied thereto, said outer circumferential edge having an indented region situated at the apically linked region between adjacent crescent shaped portions, and the method includes placing a plurality of said electrodes close to the periphery of the wound by selecting to place either the inner circumferential edges of said plurality of said electrodes adjacent to the wound, or the indented region of the outer circumferential edges of said plurality of said electrodes adjacent to the wound.

28. An electrode arrangement for applying electrical signals to said skin, said electrode arrangement comprising:
a flexible electrically non-conductive substrate;
at least one electrode provided on said substrate for applying electrical signals to the skin;
electrically conductive gel provided on said at least one electrode to provide an electrical path to said skin; and
retaining means for holding said gel in a plurality of sub regions across said at least one electrode so as to prevent said gel from moving from a sub region laterally under pressure.

29. An electrode arrangement according to claim 28, wherein said retaining means comprises a plurality of walls of flexible material extending from said at least one electrode in a two dimensional pattern over said at least one electrode, said walls define said sub regions, and said gel is provided on said at least one electrode between said pattern of walls.

30. An electrode arrangement according to claim 29, wherein said walls are formed of electrically insulating material.

31. An electrode arrangement for applying electrical signals to said skin, said electrode arrangement comprising:
a flexible electrically non-conductive substrate;
at least one electrode provided on said substrate for applying electrical signals to the skin;
a plurality of walls of flexible material extending from said at least one electrode in a two dimensional pattern over said at least one electrode; and
electrically conductive gel provided on said at least one electrode between said pattern of walls to provide an electrical path to said skin, said gel being laterally contained by said pattern of walls.

32. An electrode arrangement according to claim 31, wherein said gel is highly resistive to overcome skin resistance.

33. An electrode arrangement according to claim 31, wherein said walls are formed of electrically insulating material.

34. A wound treatment arrangement for treating a wound in the skin of a patient, the wound treating arrangement comprising:
a compression dressing arrangement for application to the limb of the patient to apply pressure thereto; and
a flat electrode arrangement for applying electrical signals to said skin, said electrode arrangement comprising: a flexible electrically non-conductive substrate; and at least one electrode provided on said substrate for applying electrical signals to the skin;
wherein said electrode arrangement is adapted for arrangement on said skin adjacent said wound under said compression dressing arrangement, and a high resistance is connected in series with said at least one electrode.

35. A wound treatment arrangement according to claim 34, wherein the high resistance comprises a highly resistive gel provided on a surface of said at least one electrode.

36. A wound treatment according to claim 34, wherein the high resistance comprises a plurality of elements of high resistivity distributed across at least a part of the surface of said at least one electrode for conduction of the electrical signal from said at least one electrode to the skin, wherein the elements of high resistivity are interspaced by portions of relatively electrically insulating material such that the elements of high resistivity are electrically insulated from one another.

37. A wound treatment arrangement according to claim 36, wherein the elements of high resistivity comprise highly resistive gel.

38. A wound treatment arrangement according to claim 36, wherein the portions of relatively electrically insulating material comprise rubber.

39. An electrode arrangement for applying electrical signals to said skin, said electrode arrangement comprising:
a flexible electrically non-conductive substrate;
at least one electrode provided on said substrate for applying electrical signals to the skin; and
a layer of electrically conductive gel lying over said at least one electrode and extending beyond said at least one electrode to cover a boundary region of said substrate for adhering said electrode arrangement to the skin and for providing an electrical path from said at least one electrode to the skin, wherein the thickness of said layer of gel is substantially smaller than the lateral length of said boundary region extending beyond said at least one electrode.

40. An electrode arrangement according to claim 39, wherein said substrate and said gel form a seal around said electrode to prevent the ingress of water to said at least one electrode.

41. An electrode arrangement according to claim 39, including at least one electrical connector lead connected to said at least one electrode, wherein said substrate and said gel is adapted to seal said skin under said gel, said electrode, and said at least one connector lead in the region of connection to said at least one electrode to prevent the ingress of water to an electrical path from said connector to said skin.

42. An electrode arrangement according to claim 39, wherein said boundary portion has a length of 4-8 mm.

43. An electrode arrangement according to claim 40, wherein the formed seal is arranged to effectively seal under a pressure of 50-60 mm Hg constant pressure for up to 7 to 9 days.

44. A wound treatment arrangement for treating a wound in the skin of a patient, the wound treating arrangement comprising:
a compression dressing arrangement applied to the limb of the patient and applying pressure thereto; and
a flat electrode arrangement for applying electrical signals to said skin, said electrode arrangement comprising: a flexible electrically non-conductive substrate; and
at least one electrode provided on a first region of a first surface of said substrate for applying electrical signals to the skin;
wherein said at least one electrode is arranged on said skin adjacent said wound under said compression dressing arrangement, said substrate includes an extended portion comprising at least one electrically conductive region, the or each electrically conductive region is in electrical contact with a respective electrode to form a respective connector to said electrode, and the at least one connector extends under and out from said compression dressing arrangement to enable electrical signals to be applied to said skin.

45. A wound treatment arrangement according to claim 44, including an electrical generator apparatus attached to said compression dressing arrangement and connected to said at least one connector for generating electrical signals to be applied to said skin.

46. A wound treatment arrangement according to claim 44, wherein said at least one electrode comprises a plurality of interconnected electrically conductive elements to form a two-dimensional electrically conductive path across at least a portion of the first region of the first surface of the substrate.

47. A wound treatment arrangement according to claim 46, wherein the plurality of interconnected electrically conductive elements are arranged in a matrix to allow flexion of the electrode arrangement.

48. A wound treatment arrangement according to claim 46, wherein the plurality of interconnected electrically conductive elements are arranged in a mesh pattern to allow flexion of the electrode arrangement.

49. A wound treatment arrangement according to claim 44, wherein electrically conductive gel is provided on said at least one electrode to provide an electrically conductive path to the skin.

50. A wound treatment arrangement according to claim 45, wherein said at least one electrode comprises at least three electrodes and said at least one connectors comprises at least three respective connectors, wherein the electrical generator apparatus is adapted to switch current to flow between different electrodes of the at least three electrodes.

51. A wound treatment arrangement according to claim 44, wherein said first region of said substrate has a part annular or crescent shape.

52. A kit of parts for treating a wound in the skin of a patient, the kit comprising:
a compression dressing arrangement for application to the limb of the patient to apply pressure thereto; and
a flat electrode arrangement for applying electrical signals to said skin, said electrode arrangement comprising: a flexible electrically non-conductive substrate; and at least one electrode provided on a first region of a first surface of said substrate for applying electrical signals to the skin; wherein said at least one electrode is adapted for arrangement on said skin adjacent said wound under said compression dressing arrangement, said substrate includes an extended portion comprising at least one electrically conductive region, the or each electrically conductive region is in electrical contact with a respective electrode to form a respective connector to said electrode, and the at least one connector is adapted to extend under and out from said compression dressing arrangement to enable electrical signals to be applied to said skin.

53. A wound treatment arrangement for treating a wound in the skin of a patient, the wound treating arrangement comprising:
a compression dressing arrangement applied to the limb of the patient and applying pressure thereto; and
a flat electrode arrangement for applying electrical signals to said skin, said electrode arrangement comprising: a flexible electrically non-conductive substrate; and at least one electrode provided on said substrate for applying electrical signals to the skin;
wherein said electrode arrangement is arranged on said skin adjacent said wound under said compression dressing arrangement, and a high resistance is connected in series with said at least one electrode.

54. A wound treatment arrangement according to claim 53, wherein the high resistance comprises a highly resistive gel provided on a surface of said at least one electrode.

55. A wound treatment according to claim 53, wherein the high resistance comprises a plurality of elements of high resistivity distributed across at least a part of the surface of said at least one electrode for conduction of the electrical signal from said at least one electrode to the skin, wherein the elements of high resistivity are interspaced by portions of relatively electrically insulating material such that the elements of high resistivity are electrically insulated from one another.

56. A wound treatment arrangement according to claim 55, wherein the elements of high resistivity comprise highly resistive gel.

57. A wound treatment arrangement according to claim 55, wherein the portions of relatively electrically insulating material comprise rubber.

58. A kit of parts for treating a wound in the skin of a patient, the kit comprising:
a compression dressing arrangement for application to the limb of the patient to apply pressure thereto; and
a flat electrode arrangement for applying electrical signals to said skin, said electrode arrangement comprising: a flexible electrically non-conductive substrate; and at least one electrode provided on said substrate for applying electrical signals to the skin; wherein said electrode arrangement is adapted for arrangement on said skin adjacent said wound under said compression dressing arrangement, and a high resistance is connected in series with said at least one electrode.
